(19) United States
(12) Patent Application Publication
(10) Pub. No.: US 2004/0265323 A1
(43) Pub. Date: Dec. 30, 2004

(54) Compositions comprising pathogen elicited epithelial chemoattractant (eicosanoid hepoxilin A3), inhibitors thereof and methods of use thereof

(76) Inventors: Beth A. McCormick, Charlestown, MA (US); Randall Mrsny, Cardiff (GB); James L. Madara, Chicago, IL (US)

Correspondence Address: FROMMER LAWRENCE & HAUG, 745 FIFTH AVENUE- 10TH FL., NEW YORK, NY, 10151, US

(21) Appl. No.: 10/848,472

(22) Filed: May 17, 2004

Related U.S. Application Data
(60) Provisional application No. 60/471,003, filed on May 16, 2003.
(60) Provisional application No. 60/480,982, filed on Jun. 23, 2003.

(51) Int. Cl. A61K 39/00; A61K 39/02

(52) U.S. Cl. 424/185.1; 424/190.1

(57) ABSTRACT

The present invention relates to the use of eiconosoid hepoxilin A3 to modulate epithelial transmigration of inflammatory cells. The present invention further relates to pharmaceutical compositions comprising native hepoxilin A3, hepoxilin A3 analogs, or specific inhibitors thereof, as well as inhibitors of the biosynthetic pathway of hepoxilin A3. Compositions and methods of the present invention are useful for enhancing cellular immunity or treating disorders associated with epithelial inflammation.

### RELATED APPLICATIONS/PATENTS & INCORPORATION BY REFERENCE

This application claims priority to U.S. Provisional Application Ser. No. 60/471,003, filed on May 16, 2003 and U.S. Provisional Application Ser. No. 60/480,982, filed on Jun. 23, 2003.

### STATEMENT OF POTENTIAL GOVERNMENT INTEREST

The United States government may have certain rights in this invention by virtue of grant number DK 57654 from The National Institute of Diabetes & Digestive & Kidney Diseases.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference, and may be employed in the practice of the invention. More generally, documents or references are cited in this text, either in a Reference List before the claims, or in the text itself, and, each of these documents or references (“herein cited references”), as well as each document or reference cited in each of the herein cited references (including any manufacturer's specifications, instructions, etc.), is hereby expressly incorporated herein by reference.

### FIELD OF THE INVENTION

The present invention relates to compositions and methods for modulating epithelial transmigration of inflammatory cells (e.g., neutrophils, such as polymorphonuclear leukocytes (“PMNs”)). Hepoxilin A3, an arachidonic acid metabolite (known in the art as pathogen-elicited epithelial chemoattractant) produced through the enzymatic action of 12-lipoxygenase, is secreted from the apical epithelium in response to pathogen invasion. It has now been shown that its action stimulates neutrophil transmigration across polarized epithelium. Identification of the novel hepoxilin A3 function presents new therapeutic opportunities for treating a number of disorders associated with inflammation of epithelium including, but not limited to, inflammatory bowel disease. Immunosupression by inhibiting neutrophil movement can promote therapeutic effects in patients suffering from epithelial inflammation. Conversely, stimulating neutrophil transmigration can increase the potency of immune responses to gastrointestinal infections.

### BACKGROUND OF THE INVENTION

Infectious pathogens continually confront epithelial barriers of the body, such as those of the gastrointestinal, respiratory and reproductive tracts. Through a series of selective toll-like receptor pathway activation events, a variety of white blood cells are recruited to these areas of pathogen challenge (Imler and Hoffman, 2000). An effective host defense response initiated by these white cells against the multitude of mucosal pathogens confronted by these barriers is paramount for survival.

Polymorphonuclear leukocytes (“PMNs”) represent a class of white cells that are critical in defending against infections by bacterial and parasitic pathogens at mucosal surfaces. Transepithelial migration of white cells such as PMNs to the site of infection is critical for effective innate immune responses to mucosal pathogens. PMN migration is an early event in the mechanism of epithelial perturbation, which includes one or more of the following events: abnormal fluid and electrolyte transport, specific epithelial barrier dysfunction, and ultimately mucosal breakdown. Often, these perturbations lead to chronic and episodic inflammatory conditions such as gastritis, diverticulitis, pulmonary complications in cystic fibrosis, chronic obstructive pulmonary disease, infectious colitis, bronchitis, asthma, Crohnis disease, nephritis, alveolitis, intestinal ulcers, idiopathic AIDS enteropathy, gastroenteritis, ischemic diseases, and glomerulonephritis. The efficacy of existing therapy for epithelial inflammation, such as methotrexate or corticosteroids, is highly unsatisfactory, partially due to a high toxicity which produces severe, adverse effects such as bone-weakening and systemic immuno-suppression (Physician's Desk Reference, 1987). Even under ideal bioavailability conditions, the existing treatments fail to mechanistically target epithelial inflammation.

Previous studies have identified secreted cytokines and lymphokines that establish a concentration gradient critical for the activation and recruitment of PMNs to such sites of mucosal infection (McCormick, et al., 1993; McCormick et al., 1995; McCormick et al., 1998). These chemokines attract PMNs from the bloodstream and/or from local submucosal sites in a manner that establishes a population that resides between adjacent epithelial cells. However, this gradient of known PMN chemokines does not provide for the continued movement of PMNs across the epithelia where they can engage pathogens in the intestinal lumen. This final epithelial barrier, established by the presence of the tight junction, represents an occlusive structure that impedes the movement of hydrophilic molecules larger than 250 Da (Nusrat et al., 2000). Identification of a factor responsible for the transmigration of PMNs across this last barrier for entry into the intestinal lumen has been an important, unanswered question of epithelial pathobiology.

An example where these principles of epithelial pathobiology play a role in the treatment of human disease is in the colonization of the human small intestine by Salmonella typhimurium, a leading cause of diarrhea worldwide. Following infection by this pathogen, mucosal inflammation leads to infiltration of PMNs (primarily neutrophils) into the intestinal epithelium in an event that culminates in the formation of an intestinal crypt abscess (Kumar et al., 1982; Rout et al., 1974; McGovern and Slavutin, 1979). The action of these PMNs on the epithelium and the subsequent loss of barrier function are thought to be key events in mediating the clinical manifestations of S. typhimurium-induced enteritis.

This model of S. typhimurium infection was used previously to demonstrate that the intestinal epithelium is not merely a barrier to PMN movement but rather, in response to this enteric pathogen, intestinal epithelial cells direct PMN movement via the polarized secretion of chemokines. Specifically, S. typhimurium activates the transcription factor NF-κB resulting in the epithelial synthesis and basolateral release of a potent PMN chemokine, interleukin-8 (IL-8) (McCormick et al., 1995). Such basolateral secretion of IL-8 recruits PMNs through the matrix (lamina propria) to the subepithelial space but is not involved in PMN migration across epithelial tight junctions, the final step of crypt abscess formation. Instead, this final step of PMN movement across the epithelium has been shown to be directed by NF-κB-independent release of a soluble bioactive agent from the apical surface of the epithelium, the identity of which has remained elusive.

Identification of the biomolecule(s) responsible for this activity would present new therapeutic opportunities for treating a number of disorders associated with inflammation of epithelium which include, but are not limited to inflammatory bowel disease. Promoting immunosupression through inhibition of neutrophil movement can bring therapeutic benefits to patients suffering from epithelial inflammation. Conversely, stimulating neutrophil transmigration can increase the potency of immune responses to gastrointestinal infections.

### SUMMARY OF THE INVENTION

Eicosanoid hepoxilin A3, an arachidonic acid metabolite (known in the art as pathogen-elicited epithelial chemoattractant) produced through the enzymatic action of 12-lipoxygenase, is secreted from the apical epithelium in response to inflammation (e.g., inflammation caused by pathogen invasion or disease). It has now been shown that hepoxilin A3 stimulates inflammatory cell transmigration across polarized epithelium.

Hepoxilin A₃ is secreted from the apical surface of epithelial cells stimulated with, for example, pathogens (e.g., Salmonella typhimurium), and functions to draw inflammatory cells, via the establishment of a gradient across the epithelial tight junction complex. This is a new function of hepoxilin A3, which was previously implicated in potentiating glucose-dependent insulin secretion, modulating synaptic neurotransmission in rat hippocampus (Pace-Asciak et al., 1999) and regulating processes mediated by intracellular calcium levels inside the neutrophil (U.S. Pat. No. 5,616,607).

Interruption of the synthetic pathway of hepoxilin A₃ blocks the apical release of hepoxilin A₃ and, as a direct effect, the transmigration of migratory cells induced by inflammation arising from, for example, pathogenic infection (e.g., S. typhimurium infection). Importantly, cellular transmigration is blocked in a physiological basolateral-to-apical direction. This is in contrast to other metabolites of arachidonic acid, for example A4, which have been shown to affect cellular transmigration in a nonphysiologically directed apical-to-basolateral direction (U.S. Pat. No. 6,458,839).

Use of hepoxilin A3 is important for developing therapies directed to the treatment of acute and chronic inflammation of epithelium, including “columnar epithelium.” The term “columnar epithelium” includes, but is not limited to, epithelium of the intestine, kidney, stomach, liver, thyroid, trachea, lung, gall bladder, urinary bladder, bile ducts, pancreatic ducts, liver, testicles, uterus and skin. For example, inhibition of hepoxilin A3 will reduce transmigration of inflammatory cells and provide topical treatment of acute and chronic inflammation of the skin. Conversely, use of hepoxilin A3 to stimulate inflammatory cell transmigration at a site of infection including, but not limited to, respiratory, intestinal and skin infection, or to a site of physical and/or chemical epithelial injury, can enhance cellular immunity.

Accordingly, the present invention relates to compositions and methods for modulating epithelial migration of inflammatory cells including, but not limited to, monocytes, lymphocytes, eosinophils, neutrophils and basophils. Methods of the present invention comprise administration of hepoxilin A3, hepoxilin A3 analogs or inhibitors of hepoxilin A3 activity or synthesis, as desired to modulate inflammatory responses in the epithelium, for example, in gastrointestinal, skin and respiratory tissues. Preferably, modulation by hepoxilin A3 is of inflammatory cell movement across epithelium (i.e., transmigration), but the invention is not so limited, and can be more generally directed to modulation of inflammatory cell migration in a desired direction.

In one embodiment, the present invention relates to compositions comprising pharmaceutical formulations of hepoxilin A3, or hepoxilin A3 analogs that likewise stimulate inflammatory cell recruitment. Stimulating inflammatory cell transmigration can increase the potency of immune responses, for example, at a site of gastrointestinal, respiratory or skin infection and/or injury.

In yet another embodiment, the present invention comprises a method of identifying compounds or compositions that mimic, exceed or increase hepoxilin A3 activity, for example hepoxilin A3 analogs. Accordingly, methods of the present invention further relate to screening for compounds that mimic or exceed hepoxilin A3 activity or induce its synthesis.

In yet another embodiment, the present invention comprises compositions that block the synthesis or activity of hepoxilin A3, such as inhibitors of 12-lipoxygenase. Inhibition of hepoxilin A3 activity is useful for treating a number of disorders associated with inflammation of epithelium including, but not limited to, inflammatory bowel disease, cystic fibrosis and chronic obstructive pulmonary disease. Promoting immunosupression by inhibition of neutrophil movement can bring therapeutic benefit to patients suffering from intestinal, lung and skin inflammation.

In yet another embodiment, the present invention comprises a method of treating inflammation in a subject in need thereof comprising administering to the subject a pharmaceutical composition comprising an inhibitor of hepoxilin A3 synthesis or activity in an amount sufficient to reduce inflammatory cell accumulation.

Methods of the present invention comprise, for example, methods of blocking PMN transmigration by inhibiting hepoxilin A3 activity including, but not limited to, application of monoclonal antibodies, small molecules, antisense oligonucleotides or ribozymes, targeting either hepoxilin A3 or biomolecules involved in its synthesis (e.g., 12-lipoxygenase).

In yet another embodiment, the present invention comprises a method of reducing inflammation in a subject in need thereof comprising administering to the subject a pharmaceutical composition comprising an inhibitor of hepoxilin A3 secretion in an amount sufficient to reduce inflammatory cell migration. Preferably, the inhibitor reduces the activity of a hepoxilin A3 transporter. The transporter can be a MDR protein pump, and the inhibitor can be, but is not limited to, cyclosporine A, verapamil, ethacrynic acid, probenecid. Preferably, the transporter is MRP-2.

In yet another embodiment, the present invention comprises a method of treating inflammation in a subject in need thereof comprising administering to the subject a pharmaceutical composition comprising an inhibitor of hepoxilin A3 secretion in an amount sufficient to reduce inflammatory cell accumulation.

In yet another embodiment, the present invention comprises a method of identifying compounds or compositions that inhibit hepoxilin A3 activity or synthesis. Accordingly, methods of the present invention further relate to screening for compounds that inhibit A3 activity or synthesis.

In this disclosure, “comprises,” “comprising,” “containing” and “having” and the like can have the meaning ascribed to them in U.S. Patent law and can mean, “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### BRIEF DESCRIPTION OF THE DRAWINGS

The following Detailed Description, given by way of example, but not intended to limit the invention to specific embodiments described, may be understood in conjunction with the accompanying drawings, incorporated herein by reference, in which:

FIG. 1 depicts PMN transepithelial migration across the intestinal epithelium during acute phases of S. typhimurium infection.

FIG. 2 depicts PEEC bioactivity, which is enriched by hydrophobic surface chromatography using a methanol gradient. FIG. 2A depicts the majority of PEEC bioactivity, measured by PMN transepithelial migration, partitioned to the methanol elution fraction. “WT” represents the PMN response to wild type S. typhimurium. FIG. 2B depicts PEEC obtained from the methanol eluted fractions, which also elicits PMN intracellular Ca²⁺ mobilization.

FIG. 3 depicts identification of PEEC. FIG. 3A depicts absorbance (at 214 nm) of a 50-55 minute preparation (obtained from semi-preparative HPLC methods). FIG. 3B depicts analytical HPLC of a collected PEEC fraction, showing one prominent peak with a retention time of 18.5 min. FIG. 3C depicts the negative ion mode mass profile of an 18.5 minute preparation, having a prominent peak at 335 and a secondary peak at 693. FIG. 3D depicts an analytical HPLC profile and negative ion electrospray MS analysis of PEEC material following overnight incubation in acid.

FIG. 4 depicts the PMN transepithelial migration and intracellular Ca²⁺ mobilization elicited by hepoxilin A3. FIG. 4A depicts imposed gradients of hepoxilin A₃ (0.5-5 μg/ml), which induce PMN transepithelial migration in a dose dependent fashion. FIG. 4B depicts an increase in intracellular Ca²⁺ in PMN treated with hepoxilin A₃ (5 μg/ml), while 12- (R) HETE, another metabolite of the 12-lipoxygenase pathway, failed to cause an increase in Ca²⁺ mobilization. FIG. 4C depicts the effect of disrupting the hepoxilin A₃ gradient (by adding 5 μg/ml of this eicosinoid to the basolateral compartment) on Salmonella-induced PMN transepithelial migration. Control stands for either wild type S. typhimurium, recombinant SipA (40 μg/ml), hepoxilin A₃ (5 μg/ml), or fMLP (100 mM) in the absence of hepoxilin A₃ administration to the basolateral compartment. Data are plotted as the percent of the control response and represent one of at least two experiments performed in triplicate. Statistical analysis (Students t test) of the raw data reveal differences, * P<0.025 and ** P<0.010, as compared to their respective control. FIG. 4D depicts the rate of hepoxilin A₃ degradation at the basolateral surface. Decay was exponential and T_{1/2} values were determined by analyzing the slope of the best-fit line to a log-linear analysis of each data set. FIG. 4E depicts hepoxilin A₃ recovered in the basolateral compartment after the addition of 4500 pmole to the apical compartment. Data points are from mean values of 2 studies and indicate that the amount of hepoxilin A₃ recovered at the basolateral surface is low (N=2).

FIG. 5 depicts PMN transepithelial migration. FIG. 5A depicts PMN transepithelial migration induced by S. typhimurium, which demonstrated a dose dependent decrease in the presence of a 12-lipoxygenase inhibitor (baicalein), whereas S. typhimurium-induced PMN transepithelial migration showed no inhibition in the presence of a specific 5-lipoxygenase inhibitor (caffeic acid). Black bars represent monolayers stimulated with S. typhimurium; open bars represent monolayers with imposed gradients of the potent PMN chemoattractant, fMLP; * P<0.025; Student's t test. FIG. 5B depicts T84 cell monolayers infected with S. typhimurium in the absence and presence of the 12-lipoxygenase inhibitor, baicalein. Under conditions of baicalein treatment, S. typhimurium induction of PEEC is dramatically reduced (* P<0.05; Student's t test). FIG. 5C depicts the effect of 12-lipoxygenase inhibition on the ability of P. aeruginosa to induce PMN transepithelial migration and elicit apical hepoxilin A₃ secretion. The black bars and (−), represent the control PMN transepithelial migration or apical release of hepoxilin A₃ in the absence of 12-LO inhibition. The gray bars and (+) represent the experimental outcome for PMN transepithelial migration and apical release of hepoxilin A₃ in the presence of the 12-lipoxygenase inhibitor. Data are expressed as mean±SD of triplicate samples for each condition and represent one out of three experiments performed with comparable results. * P<0.01; Student's t test.

FIG. 6 depicts the route of potential synthesis and observed activities of arachidonic acid and some its metabolites for the stimulation of PMN transepithelial migration. Arrows depict associations of functional moieties and not necessarily synthetic pathways. Arachidonic acid can be converted by lipoxygenase activities with specificities that include the either the 5, 8, 12 or 15 carbon. Some molecules have multiple oxidation sites (i.e. more than one lipoxygenase activity is required) while others, such as the hepoxilins, condense adjacent hydroxyl groups into a peroxide moiety.

FIG. 7 depicts histopathology of human intestinal xanografts. Each panel is a representative example of the histopathology of the intestinal epithelium (longitudinal section stained with hematoxylin and eosin) inoculated with S. typhimurium in the absence and presence of 1 μM baicalein. FIG. 7A depicts normal intestinal epithelium of a xenograft injected with buffer in the absence of S. typhimurium. FIG. 7B depicts the xenografts after S. typhimurium infection, presenting with a severe pathology. Noted is the profuse dissemination of red blood cells (black arrowheads) and a substantial PMN infiltrate (asterisk), within the mucosa and sub-mucosa. Also shown is an early stage of the formation of a crypt abscess (white open arrow). FIG. 7C depicts the histopathology of the xenografts infected with S. typhimurium in the presence of 1 μM baicalein, which is significantly less severe. These xenografts exhibited only minimal bleeding and PMN were seldom observed in the mucosa or epithelium. FIG. 7D depicts the effects of S. typhimurium on the severity in human intestinal xenografts in the absence and presence of 12-lipoxygenase inhibition. Histological severity of intestinal inflammation was assessed in a blinded fashion by an expert pathologist and was ranked (0-3) for (1) epithelial cell damage, (2) congestion and edema, and (3) PMN infiltration (Triadafilopoulos et al., 1987). Data are mean±SEM of six to ten grafts per group (P<0.05; ^{a, b, c}, significant differences between buffer control and S. typhimurium; buffer control and S. typhimurium plus baicalein; and S. typhimurium in the absence and presence of baicalein, respectively).

FIG. 8 depicts therapeutic effect of baicalein on intestinal inflammation. FIG. 8A depicts SCID mice with rectal prolapse induced by injection of CD45RbHI-T cells. FIG. 8B depicts SCID mice after 2.5 weeks of buffer control or baicalein administration. The arrows point out to the specific diseased SCID mice that were treated with buffer control or baicalein after adoptive transfer of CD45RbHI T cells.

FIG. 9 depicts the effect of inhibitors of multidrug resistance on the ability of S. typhimurium to induce PMN transepithelialmigration. For these experiments, the following MDR inhibitors were used: cyclosporine A, verapamil, ethacrynic acid and probenecid. All the graphs are represented as the percent inhibition.

FIG. 10 depicts expression of MRP-2 and MRP-3 proteins. FIG. 10A depicts expression of MRP-2 after infecting T84 cells with S. typhimurium, SipA mutant strain, LPS and commercial E. coli strain, respectively. FIG. 10B depicts expression of MRP-3 after infecting T84 cells with S. typhimurium.

FIG. 11 depicts expression of MRP-2 protein in healthy, diseased and baicalein treated mice. FIG. 11A depicts MRP-2 expression in healthy controls, FIG. 11B depicts expression of MRP-2 after 6 weeks of adoptive transfer with CD45Rbhi subset of Tcells, and FIG. 11C depicts expression of MRP-2 following treatment of sick mice with baicalein.

### DETAILED DESCRIPTION OF THE INVENTION

The present invention relates to compositions and methods for modulating hepoxilin A3- stimulated migration of inflammatory cells including, but not limited to, monocytes, lymphocytes, eosinophils, neutrophils and basophils across the epithelial surface. Preferably, the inflammatory cells comprise neutrophils, such as polymorphonuclear leukocytes (“PMNs”). Methods of the present invention comprise administration of hepoxilin A3, hepoxilin A3 analogs or inhibitors of hepoxilin A3 activity or synthesis, as desired to regulate inflammatory responses in epithelial tissues, for example gastrointestinal, skin and respiratory tissues.

As used herein, the term “modulating” means regulating or controlling as necessary, through eliminating, reducing, maintaining or increasing a desired effect. The desired effect can be an effect on inflammatory cell migration or transmigration. “Modulation” of inflammatory cells refers to the ability to control, regulate, or activate a physiological response within the cells that ultimately changes the migratory state of the cells.

The term “transmigration” refers to movement of inflammatory cells across epithelium in a basolateral-to-apical direction. “Migration” refers more generally to any movement of inflammatory cells.

“Accumulation” of inflammatory cells refers to the build up of inflammatory cells during an immune response.

An “immune response” refers to the process whereby inflammatory cells are recruited from the blood to lymphoid and non-lymphoid tissues via a multifactorial process that involves distinct adhesive and activation steps. Inflammatory conditions cause the release of chemokines and other factors that, by upregulating and activating adhesion molecules on both endothelial cells and inflammatory cells, promote adhesion, morphological changes, and extravasation concurrent with chemotaxis through the tissues.

“Inflammation,” as used herein refers to an adverse immune response having a detrimental health effect in a subject. A “subject” is a vertebrate, preferably a mammal, more preferably a human. Mammals include, but are not limited to, humans, farm animals, sport animals, and pets. Inflammation can be caused, for example, by pathogenic infection, irritation or disease. Inflammation can also be caused by autoimmunity, wherein a subject's own antibodies react with host tissue or in which immune effector T cells are autoreactive to endogenous self-peptides and cause destruction of tissue.

In one embodiment, compositions and methods of the present invention promote immunosupression through reduction or inhibition of inflammatory cell migration to a site of inflammation. Immunosupression can bring therapeutic benefit to patients suffering from epithelium inflammation. Compositions and methods of the present invention that inhibit hepoxilin A3 activity or synthesis are useful for treating a number of disorders associated with inflammation of columnar epithelium, such as inflammatory bowel disease, chronic obstructive pulmonary disease and cystic fibrosis. As used herein, the term “columnar epithelium” includes, but is not limited to, epithelium of the intestine, kidney, stomach, liver, thyroid, trachea, lung, gall bladder, urinary bladder, bile ducts, pancreatic ducts, liver, testicles, uterus and skin. For example, inhibition of hepoxilin A3 activity or synthesis will reduce transmigration of inflammatory cells and provide topical treatment of acute and chronic inflammation of the skin.

Compositions that block the synthesis or activity of hepoxilin A3 can be administered to a subject in need thereof in a therapeutically effective pharmaceutical composition. Such compositions can be directed to treating inflammation of epithelium often associated with disorders including, but not limited, to enterocolitis; viral infections such as non-specific enteritis or specific viral enteritis; diverticulitis; bacterial enterocolitis, such as salmonellosis, shigellosis, campylobacter enterocolitis, or yersinia enterocolitis; protozoan infections such as amebiasis; helminthic infection; and pseudomembraneous colitis and pulmonary complications in cystic fibrosis and chronic obstructive pulmonary disease; appendicitis; atrophic gastritis; Barrett's esophagus; pneumonitis; cervicitis; chronic interstitial nephritis; colitis; colonic diverticulitis; conjunctivitis; contact dermatitis; Curling's ulcers; Cushing's ulcers; cystitis; gangrene; gingivitis; mastitis; esophagitis; pancreatitis; panniculitis; phlegmonous gastritis; glomerulonephritis; and autoimmune diseases including, but not limited to, inflammatory bowel disease, ulcerative colitis, Crohn's disease, Addison's disease, glomerulonephritis (e.g., crescentic glomerulonephritis, proliferative glomerulonephritis) and psoriasis.

As used herein, a “therapeutically effective composition” comprises a composition that blocks the synthesis or activity of hepoxilin A3 in an amount effective to decrease migration and accumulation of inflammatory cells at a site of inflammation. Alternatively, a “therapeutically effective composition” contains native hepoxilin A3, or a hepoxilin A3 analog, in an amount effective to stimulate migration and accumulation of inflammatory cells to a site of infection. An effective amount is any amount wherein the subject experiences either relief from inflammation or increased immune function resulting in the enhanced treatment of infection.

Hepoxilin A3 is a biologically active derivative of arachidonic acid. Once released from membrane lipids, arachidonic acid can be metabolized by 12-lipoxygenase to form 12(S)-hydroperoxy-eicosaletraenoic acid (HPETE). The 12(S)-HPETE can be further metabolized into hepoxilin A3 by a variety of ferroprotoporphyrin containing proteins (Pace-Asciak et al., 1990).

Accordingly, in yet another embodiment, the present invention comprises compositions that block the synthesis or activity of hepoxilin A3, such as inhibitors of 12-lipoxygenase.

Hepoxilin A3 is formed via the 12-lipoxygenase metabolism of arachidonic acid (Sutherland et al., 2000). Thus, methods of the present invention comprise the use of specific inhibitors of 12-lipoxygenase to interrupt the synthetic pathway of hepoxilin A3 production. 12-lipoxygenase is inhibited by various compounds known in the art, such as baicalein, pioglitazone, BW755c, aminogquanidine and Cinnamyl-3, 4-dihydroxy-alpha-cyanocinnamate. Inhibitors of 12-lipoxygenase can be provided individually, or in multiple combinations (e.g., baicalein together with pioglitazone), administered as a single combined dosage formulation, or as individual dosage formulations that are administered either concomitantly or sequentially. Preferably, inhibitory compositions or methods of administration comprise at least baicalein.

Methods of the present invention further comprise, for example, methods of blocking inflammatory cell migration by inhibiting hepoxilin A3 activity or synthesis, including, but not limited to, application of monoclonal antibodies, antisense oligonucleotides or ribozymes, targeting either hepoxilin A3 or biomolecules involved in its synthesis (e.g., 12-lipoxygenase).

Accordingly, the present invention comprises reducing or eliminating hepoxilin A3 activity by administration of antibodies binding to, for example, 12-lipoxygenase or other proteins contributing to the hepoxilin A3 synthetic pathway). Antibodies for use in the present invention can be raised against the desired target (e.g., 12-lipoxygenase), or an antigenic polypeptide fragment thereof, in an animal system (e.g., rabbit or mouse), by presentation together with a carrier protein, such as an albumin, or, if it is long enough (preferably, at least 25 amino acids), without a carrier. Methods of producing antibodies are well known in the art, and antibodies of the present invention can be generated using any standard production methods or techniques.

As used herein, the term “antibody” (Ab) or “monoclonal antibody” (Mab) is meant to include intact molecules as well as antibody fragments (such as, for example, Fab and F(ab′) 2 fragments) which are capable of specifically binding to target proteins (e.g., in the hepoxilin A3 synthetic pathway). Fab and F(ab′)2 fragments lack the Fc fragment of intact antibody, clear more rapidly from the circulation, and may have less non-specific tissue binding of an intact antibody (Wahl, et al., 1983). Thus, these fragments are preferred.

The antibodies of the present invention may be prepared by any of a variety of methods. For example, cells expressing 12-lipoxygenase or an antigenic fragment thereof can be administered to an animal in order to induce the production of sera containing polyclonal antibodies. In a preferred method, a preparation of the 12-lipoxygenase is prepared and purified to render it substantially free of natural contaminants. Such a preparation is then introduced into an animal in order to produce polyclonal antisera of greater specific activity.

In the most preferred method, the antibodies of the present invention are monoclonal antibodies. Such monoclonal antibodies can be prepared using hybridoma technology (Kohler, et al., 1975; Kluskens et al., 1976; Hammerling, 1981). In general, such procedures involve immunizing an animal (preferably a mouse) with, for example, 12-lipoxygenase or, more preferably, with a 12-lipoxygenase-expressing cell. Suitable cells can be recognized by their capacity to bind 12-lipoxygenase antibody. Such cells may be cultured in any suitable tissue culture medium; however, it is preferable to culture cells in Earle's modified Eagle's medium supplemented with 10% fetal bovine serum (inactivated at about 56.degree. C.), and supplemented with about 10 mu.g/l of nonessential amino acids, about 1,000 U/ml of penicillin, and about 100 mu.g/ml of streptomycin. The splenocytes of such mice are extracted and fused with a suitable myeloma cell line. Any suitable myeloma cell line may be employed in accordance with the present invention; however, it is preferable to employ the parent myeloma cell line (SP2O), available from the American Type Culture Collection, Manassas, Va. After fusion, the resulting hybridoma cells are selectively maintained in HAT medium, and then cloned by limiting dilution as described by Wands et al. (1981). The hybridoma cells obtained through such a selection are then assayed to identify clones which secrete antibodies capable of binding the antigen of interest.

Alternatively, additional antibodies capable of binding to, for example, the 12-lipoxygenase protein antigen, can be produced in a two-step procedure through the use of anti-idiotypic antibodies. Such a method makes use of the fact that antibodies are themselves antigens, and that, therefore, it is possible to obtain an antibody which binds to a second antibody. In accordance with this method, protein-specific antibodies are used to immunize an animal, preferably a mouse. The splenocytes of such an animal are then used to produce hybridoma cells, and the hybridoma cells are screened to identify clones, which produce an antibody, whose ability to bind to the protein-specific antibody can be blocked by the protein antigen. Such antibodies comprise anti-idiotypic antibodies to the specific antibody and can be used to immunize an animal to induce formation of further protein-specific antibodies.

It will be appreciated that Fab and F(ab′)2 and other fragments of the antibodies of the present invention may be used according to the methods disclosed herein. Such fragments are typically produced by proteolytic cleavage, using enzymes such as papain (to produce Fab fragments) or pepsin (to produce F(ab′)2 fragments). Alternatively, binding fragments can be produced through the application of recombinant DNA technology or through synthetic chemistry.

For in vivo use of, for example, an anti-12-lipoxygenase antibody in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art (Morrison, 1985; Cabilly, et al., U.S. Pat. No. 4,816,567; Boulianne, et al., 1984; Neuberger, et al., 1985).

Among the compounds that can inhibit the synthesis of hepoxilin A3 are antisense oligonucleotides, ribozymes, and triple helix molecules directed to proteins involved in the hepoxilin A3 synthetic pathway. Such molecules may be designed to reduce or inhibit, for example, 12-lipoxygenase activity. Techniques for the production and use of such molecules are well known to those of skill in the art.

Antisense RNA and DNA molecules act to directly block the translation of mRNA by binding to targeted mRNA and preventing protein translation. With respect to antisense DNA, oligodeoxyribonucleotides derived from the translation initiation site, e.g., between the −10 and +10 regions of the nucleotide sequence of interest, are preferred.

Ribozymes are enzymatic RNA molecules capable of catalyzing the specific cleavage of RNA. The mechanism of ribozyme action involves sequence specific hybridization of the ribozyme molecule to complementary target RNA, followed by an endonucleolytic cleavage. The composition of ribozyme molecules must include one or more sequences complementary to the target 12-lipoxygenase mRNA and must include the well known catalytic sequence responsible for mRNA cleavage. As such, within the scope of the invention are engineered hammerhead motif ribozyme molecules that specifically and efficiently catalyze endonucleolytic cleavage of RNA sequences encoding a target protein of interest.

Specific ribozyme cleavage sites within any potential RNA target are initially identified by scanning the target molecule for ribozyme cleavage sites which include the following sequence: GUA, GUU and GUC. Once identified, short RNA sequences of between 15 and ribonucleotides corresponding to the region of the target gene containing the cleavage site may be evaluated for predicted structural features, such as secondary structure, that may render the oligonucleotide sequence unsuitable. The suitability of candidate targets may also be evaluated by testing their accessibility to hybridization with complementary oligonucleotides, using ribonuclease protection assays.

Nucleic acid molecules to be used in triplex helix formation should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC.sup.+ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of guanidine residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so-called “switchback” nucleic acid molecule. Switchback molecules are synthesized in an alternating 5′-3′, 3′-5′ manner, such that they base pair with one strand of a duplex first and then the other, eliminating the necessity for a sizeable stretch of either purines or pyrimidines to be present on one strand of a duplex.

Antisense RNA and DNA molecules, ribozyme molecules and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various well-known modifications to the DNA molecules may be introduced as a means of increasing intracellular stability and half-life. Possible modifications include, but are not limited to, the addition of flanking sequences of ribo- or deoxy- nucleotides to the 5′ and/or 3′ ends of the molecule or the use of phosphorothioate or 2′ O-methyl rather than phosphodiesterase linkages within the oligodeoxyribonucleotide backbone.

Accordingly, the present invention further comprises reducing or eliminating hepoxilin A3 activity by inhibiting its secretion. Preferably, an inhibitor that reduces the activity of a Hepoxilin A3 transporter is employed to inhibit secretion. The transporter can be, for example, a P-glycoprotein or a MDR protein pump. Preferably, the transporter is MRP-2.

P-glycoprotein is a member of a superfamily of membrane proteins (i.e., the ATP-binding cassette (ABC) superfamily) that serve to transport a variety of molecules ranging from ions to proteins, across cell membranes. The family also includes the drug efflux pumps which mediate the cellular extrusion of a large variety of therapeutic drugs, a phenotype that is referred to as multidrug resistance (MDR)(for review see C. F. Higgins, Ann. Rev. Cell Biol. 8, 67 (1992). The physiological role of these transporters covers a wide spectrum of functions, varying from the transport of excretory compounds and the elimination of xenobiotics, to the mediation of an inflammatory response (Borst et al. (1999) Biochimica et Biophysica Acta, 1461:347-357; Borst et al. (2000) J.Natl.Cancer Inst. 92:1295-1305). Further, some transporters in this family have been shown to have substrate preference for negatively charged compounds such as hepoxilin A3.

MDR inhibitors, include, but are not limited to, cyclosporine A, verapamil, ethacrynic acid and probenecid. Ethacrynic acid and probenecid are known to block the MRP-2 transporter and thus prevent the efflux of substrates which use this transporter.

In yet another embodiment, the present invention relates to compositions comprising pharmaceutical formulations of hepoxilin A3, or hepoxilin A3 analogs that likewise stimulate inflammatory cell recruitment. Stimulating inflammatory cell transmigration can increase the potency of immune responses, for example, at the site of gastrointestinal, skin and respiratory infections.

Native hepoxilin A3, as well as hepoxilin A3 analogs that likewise stimulate inflammatory cell recruitment, can be administered to a subject in need thereof in a therapeutically effective pharmaceutical composition. Preferably, subjects of the present invention are mammals (e.g., any animal classified as a mammal, including humans, domestic or farm animals, and zoo sports, or pet animals, such as dogs, horses, cats, cows, etc). Most preferably, the mammal herein is a human.

Hepoxilin A3 analogs of the present invention comprise, for example, analogs having variations in structure, but retaining the essential functional activity of native hepoxilin A3 (i.e, the ability to recruit inflammatory cells). As referred to herein, a composition having “native hepoxilin A3 activity” possesses a functional ability recruit or mobilize inflammatory cells which is equivalent to that of hepoxilin A3 in its native (e.g., unmodified) form. Hepoxilin A3 analogs may exceed the physiological activity of native hepoxilin A3. Methods of analog design are well known in the art, and synthesis hepoxilin A3 analogs can be carried out according to such methods by modifying the chemical structure of the native hepoxilin A3 such that the resultant analogs exhibit enhanced selectivity to the binding grove of the hepoxilin A3 receptor and thus are able to successfully compete with the native hepoxilin A3 for the receptor binding site. These chemical modifications include, but are not limited to, substituting alternative R groups and varying the degree of saturation at specific carbon atoms of the native hepoxilin A3 molecule. Preferably, hepoxilin A3 analogs are relatively resistant to in vivo degradation, resulting in a more prolonged therapeutic effect upon administration. Assays for measuring functional activity include, but are not limited to, those described in the Examples below.

The pharmaceutical compositions can be administered by any means that achieve their intended purpose. For example, administration can be by topical, parenteral, subcutaneous, intravenous, intramuscular, intraperitoneal, transdermal, or buccal routes. Alternatively, or concurrently, administration can be by the oral route. The molecules can be administered parenterally by injection or by gradual profusion over time.

In addition to the pharmacologically active compounds, the pharmaceutical preparations can contain suitable pharmaceutically acceptable carriers comprising excipients and auxiliaries that facilitate processing of the active compounds into preparations that can be used pharmaceutically. Preferably, the preparations, particularly those preparations that can be administered orally and that can be used for the preferred type of administration, such as tablets, dragees, and capsules, and also preparations that can be administered rectally, such as suppositories, as well as suitable solutions for administration by injection or orally, contain from about 0.001 to about 99 percent, preferably from about 0.01 to about 95 percent, about 1.0 to about 90 percent, or about 10 to about 50 percent of active compound(s), together with the excipient. Standard texts, such as “Remington's Pharmaceutical Science”, 17th edition, 1985, “Hand Book of Pharmaceutical Excipients, ₄th edition 2003, incorporated herein by reference, may be consulted to prepare suitable preparations, without undue experimentation. Suitable dosages can also be based upon the text herein and documents cited herein.

The dosage administered will be dependent upon the age, sex, health, and weight of the recipient, kind of concurrent treatment, if any, frequency of treatment and the nature of the effect desired. The dose ranges for the administration of the compositions of the present invention are those large enough to produce the desired effect, whereby, for example, the acute inflammation is reduced or eliminated or ameliorated. The doses should not be so large as to cause adverse side effects, such as unwanted cross-reactions, anaphylactic reactions, and the like. Generally, the dosage will vary with the condition and extent of the disease in the patient. Counter indications, if any, immune tolerance, and other variables will also affect the proper dosage.

For instance, taking into account such factors as the age, weight, sex, species, general health/condition of the patient, the condition to be treated, timing of treatments, the LD50 of the active ingredient involved in a suitable animal mode (e.g., rodent, mice), and other known factors; and such dosages can be on the order of micrograms to milligrams such as on the order of 0.5 to 500 mg/kg, or another suitable amount, or can be computed from Examples herein, e.g., considering the average weight of a typical test animal (such as mice) and the dosages administered thereto (e.g., 100 micrograms), and thus the skilled artisan can determine dosages without undue experimentation.

The pharmaceutical preparations are manufactured in a manner that is itself known, for example, by means of conventional mixing, granulating, dragee making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example, lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethyl-cellulose, sodium carboxymethylcellulose, and/or polyvinyl pyrrolidone. If desired, disintegrating agents may be added, such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings that, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropymethyl-cellulose phthalate, are used. Dye stuffs or pigments may be added to the tablets or dragee coatings, for example, for identification or in order to characterize combinations of active compound doses.

Other pharmaceutical preparations that can be used orally include pushfit capsules made of gelatin, as well as soft, sealed capsules made of gelatin and a plasticizer, such as glycerol or sorbitol. The push-fit capsules can contain the active compounds in the form of granules that may be mixed with fillers, such as lactose, binders, such as starches, and/or lubricants, such as talc or magnesium stearate, and, optionally, stabilizers. In soft capsules, the active compounds are preferably dissolved or suspended in suitable liquids, such as fatty oils or liquid paraffin. In addition, stabilizers may be added.

Possible pharmaceutical preparations that can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules that consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances that increase the viscosity of the suspension including, for example, sodium carboxymethyl cellulose, sorbitol, and/or dextran. Optionally, the suspension may also contain stabilizers.

Additional pharmaceutical methods can be employed to control the duration of action. Controlled release preparations may be achieved by the use of polymers to complex or adsorb the NDS agents. The controlled delivery may be exercised by selecting appropriate macromolecules (for example, polyesters, polyamino acids, polyvinyl pyrrolidone, ethylenevinylacetate, methylcellulose, carboxymethylcellulose, and protamine sulfate) and the concentration of macromolecules as well as the methods of incorporation in order to control release. Another possible method to control the duration of action by controlled release preparations is to incorporate the derivatives of NDS into particles of a polymeric material such as polyesters, polyamino acids, hydrogels, poly(lactic acid) or ethylene vinylacetate copolymers. Alternatively, instead of incorporating the NDS derivatives into these polymeric particles, it is possible to entrap these derivatives in microcapsules prepared, for example, by coacervation techniques or by interfacial polymerization, for example, hydroxymethylcellulose or gelatin-microcapsules and poly(methylmethacrylate) microcapsules, respectively, or in colloidal drug delivery systems, for example, liposomes, albumin microspheres, microemulsions, nanoparticles, and nanocapsules or in macroemulsions.

The preclinical and clinical therapeutic use of the compositions and methods of the present invention to enhance cellular immunity or ameliorate acute and chronic inflammatory disorders of the intestine and lung epethilium will be best accomplished by those of skill, employing accepted principles of diagnosis and treatment. Such principles are known in the art, and are set forth, for example, in Petersdorf, R. G. (1983).

The molecules, antibodies, and compositions of the present invention, or their functional derivatives, are well suited for the preparation of pharmaceutical compositions. The pharmaceutical compositions of the invention can be administered to any subject that may experience the beneficial effects of the compounds of the invention.

Aspects of the present invention further comprise identifying compositions that induce or inhibit inflammatory cell transmigration.

Accordingly, in one embodiment, methods of the present invention comprise identifying compounds or compositions that mimic, exceed or increase native hepoxilin A3 activity, for example hepoxilin A3 analogs. Accordingly, methods of the present invention further relate to screening for compounds that mimic or exceed hepoxilin A3 activity or induce its synthesis.

In another embodiment, methods of the present invention comprise identifying compounds or compositions that inhibit hepoxilin A3 activity or synthesis. Accordingly, methods of the present invention further relate to screening for compounds that inhibit A3 activity or synthesis.

Design of assays (e.g., cell-based assays) to identify compounds effecting or possessing hepoxilin A3 activity, or inhibiting hepoxilin A3 activity, is well within the skill in the art. The end point of the assays will typically measure a physiologic effect. The physiologic effect can comprise inducing or inhibiting cellular migration (e.g., transmigration across the polarized epithelium) in response to interaction with a specific candidate or composition, referred to herein as a test compound. Where test compounds are directed to bioactive agents of the hepoxilin A3 biosynthetic pathway (e.g., 12-lipoxygenase), the end point of the assay can also measure, for example, hepoxilin A3 levels. Where it is desirable for a test compounds to compete for binding to the hepoxilin A3 receptor, standard receptor binding assays and/or competition assays can be conducted in accord with general techniques well known in the art.

Compositions to be screened include, but are not limited to, peptides made of D-and/or L-configuration amino acids (in, for example, the form of random peptide libraries; see Lam, K. S. et al., (1991, phosphopeptides (in, for example, the form of random or partially degenerate, directed phosphopeptide libraries; see, for example, Songyang, Z. et al. (1993), small or large organic or inorganic molecules or cellular extracts.

Preferably, epithelial cell lines are used in cell-based assays. Cell lines can consist of naturally occurring or engineered cells that synthesize hepoxilin A3. Such cell lines can be used to evaluate the efficacy of a test compound to inhibit or enhance hepoxilin A3 activity, as determined, for example, by measuring inflammatory cell transepithelial migration, receptor binding or biosynthesis. Preferably, PMN transmigration is measured.

Preferably, epithelial cell lines can be exposed to a test compound in a sufficient concentration and for a sufficient time as to elicit an effect, if any, on basolateral to apical cellular transepithelial migration. Cellular transmigration can be assayed by methods well known to those of skill in the art (McCormick et al., 1993).

The efficacy of a test compound, such as is identified in the foregoing assays, can be further assessed, for example, in immunodeficiency disease (SCID) mice, a model of immunosupression which is well known to those skilled in the art. In addition, the effect of inhibitors of hepoxilin A3 activity or synthesis on transmigration can be tested, for example, in SCID mice engrafted with human intestinal xenografts (Savidge et al., 1995).

In yet another embodiment, present invention relates to a method of transmitting data, for example disclosing the procedures and results of the various treatments described above, via global communications network. Global communications networks include the internet and other information networks. For example, data can be transmitted via website posting, such as by subscription or select or secure access thereto and/or via email and/or via telephone, IR, radio or television other frequency signal, and/or via electronic signals over cable and/or satellite transmission and/or via transmission of disks, cds, computers, hard drives, or other apparatus containing the information in electronic form, and/or transmission of written forms of the information, e.g., via facsimile transmission and the like. Thus, the invention comprehends a user performing according to the invention and transmitting information therefrom; for instance, to one or more parties who then further utilize some or all of the data or information, e.g., in the manufacture of products, such as therapeutics, assays and diagnostic tests etc. The invention also comprehends disks, cds, computers, or other apparatus or means for storing or receiving or transmitting data or information containing information from methods and/or use of methods of the invention. Thus, the invention comprehends a method for transmitting information comprising performing a method as discussed herein and transmitting a result thereof.

Further still, the invention comprehends methods of doing business comprising performing some or all of a herein method or use of a herein composition, and communicating or transmitting or divulging a result or the results thereof, advantageously in exchange for compensation, e.g., a fee. Advantageously the communicating, transmitting or divulging is via electronic means, e.g., via internet or email, or by any other transmission means herein discussed. Thus, the invention comprehends methods of doing business involving the compounds, the compositions and methods of the invention.

The following examples are provided as a further description of the invention, and to illustrate but not limit the invention.

### EXAMPLES

### Example 1

### S. typhimurium-epithelial Interactions

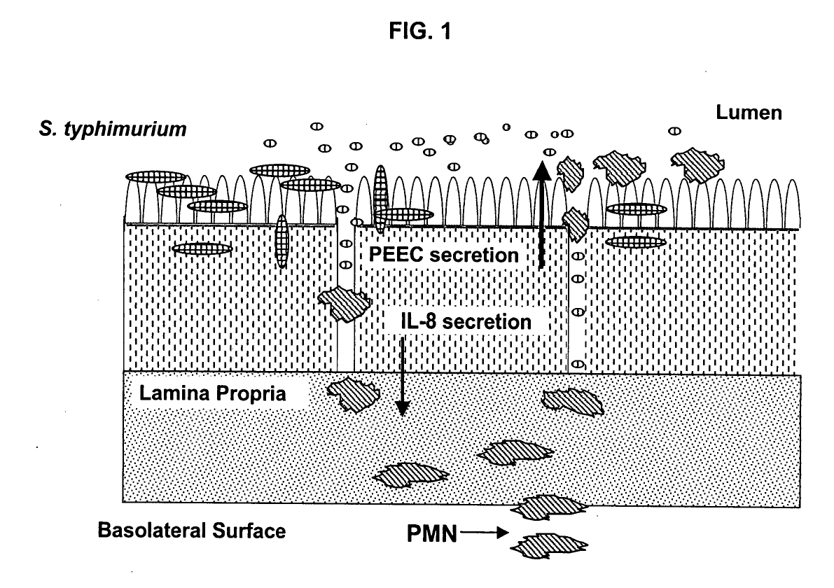

Salmonella typhimurium can orchestrate PMN movement towards and across the intestinal epithelium by activation of epithelial-derived proinflammatory pathways. Attachment of S. typhimurium to the apical membrane of enterocytes results in basolaterally directed secretion of IL-8 that recruits PMN into the subepithelial compartment. In conjunction with basolateral IL-8 release, is the apical secretion of PEEC, an essential chemoattractant that guides PMN across the epithelia into the lumenal compartment. Thus, the establishment of gradients by both IL-8 and PEEC are perceived to be required for the movement of PMN first to the subepithelial and finally the luminal compartment, respectively (FIG. 1). The absolute insufficiency of basolateral chemokines alone to direct such PMN transmigration is illustrated by the many stimuli such as purified flagellin, TNFα or carbachol that potently induce these chemokines but do not result in PMN movement across the epithelium (Gewirtz et al., 2001).

### Example 2

### Characteristics of S. typhimurium-induced PEEC Released from T84 Cells

Crude preparations of PEEC were collected from the apical surface of polarized T84 cell monolayers following infection with S. typhimurium SL1344 as previously described (McCormick et al., 1998). For these experiments, T84 intestinal epithelial cells (passages 45 to 65) were grown in a 1:1 mixture of Dulbecco-Vogt modified Eagles medium and Ham's F-12 medium supplemented with 15 mM Hepes buffer (pH 7.5), 14 mM NaHCO3, 40 mg/liter penicillin, 8 mg/liter ampicillin, 90 mg/liter streptomycin and 5% newborn calf serum. Polarized monolayers of T84 cells were formed and maintained on 0.33 cm² ring-supported collagen-coated polycarbonate filters (Costar Corp., Cambridge, Mass.) as previously described (Madara et al., 1992) with recently detailed modifications (Dharmsathaphorn and Madara, 1990). T84 cell monolayers reached a steady-state resistance 4 to 6 days after plating with some variability largely related to cell passage number. Cell culture inserts of inverted monolayers, used to study transmigration of PMN in the physiological basolateral-to-apical direction, were constructed as previously described (Madara et al., 1992; Dharmsathaphorn and Madara, 1990; Nash et al., 1991).

To study the effect of PEEC on PMN transmigration, T84 cells were infected with the wild type S. typhimurium strain SL1344, and its isogenic derivative VV341, which is rendered entry deficient by deletion of the hilA locus (Lee and Falkow, 1990; Hueck et al, 1995). For infecting T84 cells, bacteria were grown under O₂-limited conditions as originally described (Lee and Falkow, 1990). Under these conditions, bacteria were in late logarithmic phase of growth, corresponding regularly to 5-7×10⁸ colony forming units (CFU).

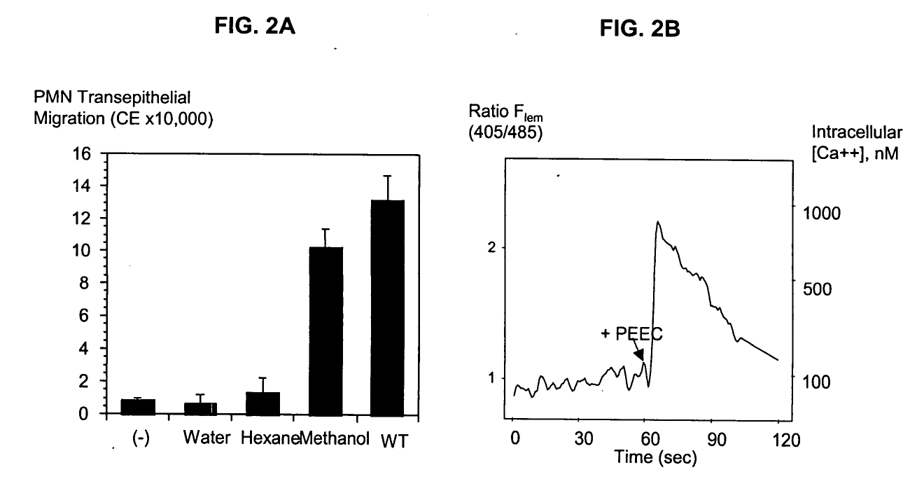

Further, crude PEEC preparations were collected from postinfected T84 cells monolayers. Samples were first passed through an Amicon ultra-filtration apparatus (Millipore Corp.; Bedford, Mass.) fitted with a 2,000 Da cut-off membrane. Filtrate components were bound to a Backerbond spe™ octadecyl extraction column (J. T. Baker; Philipsburg, N.J.) that was subsequently eluted with water, hexane, and finally methanol. The methanol fraction was dried under vacuum and resuspended in 50:50 (vol:vol) methanol:2 mM Tris HCl (pH 7.5) and injected onto a Vydac C18 (10 μm; 300 Å) semi-preparative column (10×250 cm) equilibrated with 2 mM Tris-HCl (pH 7.5). A methanol gradient of 1-10% over 10 minutes, then 10-60% over 25 minutes, followed by 60-100% over 45 minutes (all at room temperature) was used to isolate active PEEC fractions. Active fractions, having no detectable absorbance at 280 nm and weak absorbance at 214 nm, were analyzed using a Genesis C18 (4 μm, 120 Å) analytical HPLC column (4.6×150 mm) equilibrated with 5 mM triethylamine/acetic acid (pH 7.2). A peak of PEEC activity that demonstrated an ability to generate an increase in PMN cytosolic Ca²⁺ concentration and induce transmigration of PMNs across naïve T84 cell monolayers was identified. (FIG. 2A and B).

### Example 3

### Identification of PEEC

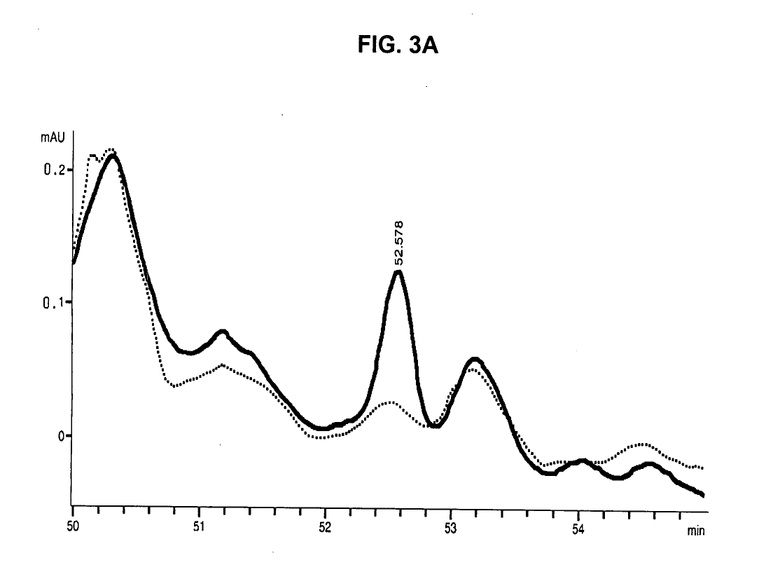

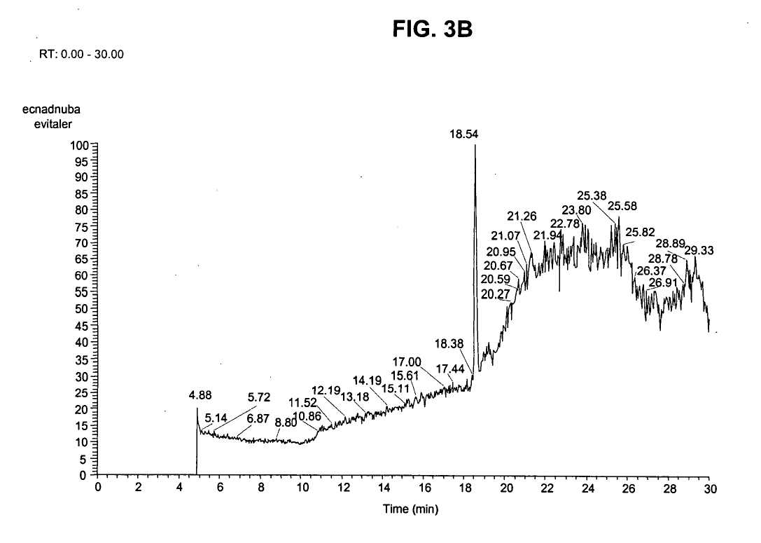

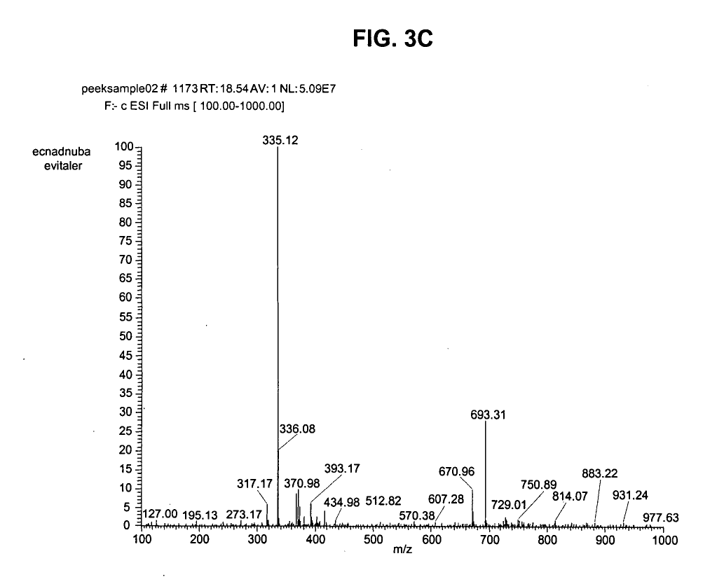

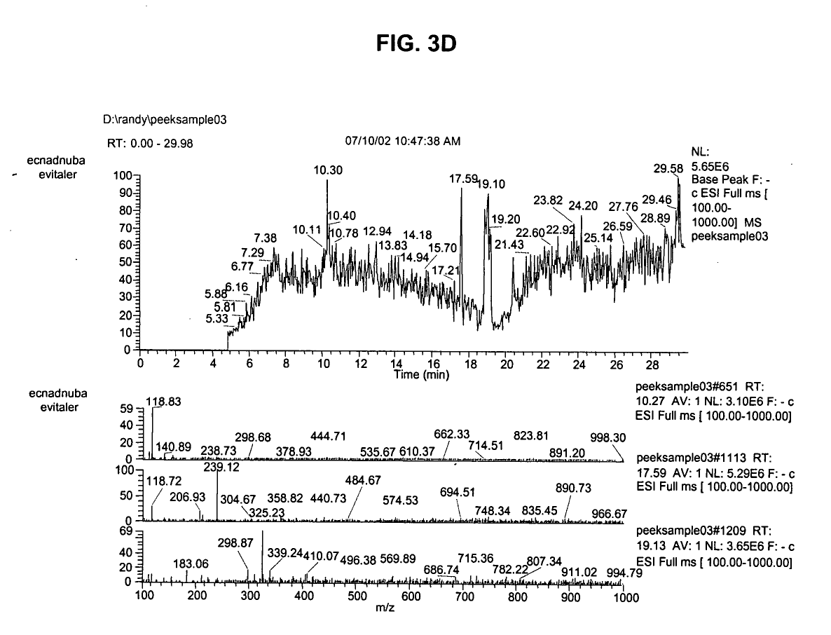

Activity-enriched preparations of PEEC were concentrated and separated using a semi-preparative HPLC method (FIG. 3A). One the fractions collected (at 52.6 min) had a potent PEEC activity. Preparations of PEEC analyzed in this way suggested a low level of PEEC was released into the apical compartment of T84 monolayer cultures under baseline conditions and that the amount of PEEC released was greatly enhanced by the presence of pathogenic S. typhimurium. Spent media obtained from incubations of T84 monolayers with S. typhimurium strain VV341, an isogenic derivative of SL1344, which is rendered entry deficient by deletion of the hilA locus and fails to incite the release of PEEC activity from T84 monolayers, was used as a control. Incidentally, strain VV341 also does not secrete SipA or any of the S. typhimurium pathogenicity island-1 effector proteins (Lee and Falkow, 1990; Lee et al., 2000). HPLC of comparable PEEC samples isolated from T84 monolayers incubated with the VV341 strain, rather than the SL1344 strain of S. typhimurium, demonstrated a much reduced absorbance peak at 52.6 min (FIG. 3A). Analytical HPLC of the collected 52.6 min peak demonstrated one major component (FIG. 3B). HPLC/electrospray mass spectrometry (MS) using negative ion mode identified a prominent mass of 335 and a less prominent mass of 694 (FIG. 3C). Tandem MS analysis demonstrated the 694 mass to be consistent with a Na⁺ salt dimer of the monomer (335) mass.

A scan of potential molecules having this mass revealed several possibilities of the eicosanoid class of arachadonic acid metabolites, which are produced through the actions of lipoxygenase (LO) that had been shown to activate or chemoattract PMNs. Molecules of the 5-LO, 8-LO, 12-LO and 15-LO pathways of arachadonic metabolism were analyzed by LC/MS breakdown patterns and compared to PEEC. These data revealed an excellent correlation between the 12-LO pathway metabolite hepoxilin A₃ and PEEC. Methylation of PEEC samples and hepoxilin A₃ also provided similar ion spectra (data not shown). A number of other arachidonic acid metabolites previously shown to affect inflammatory events, including LTB4, were also examined by LC/MS and compared to PEEC, but failed to show similar retention by analytical HPLC to that of authentic PEEC (data not shown). PEEC activity was also observed to have other chemical properties similar to hepoxilin A₃, such as being labile in the presence of acetonitrile/0.1% trifluoroacetic acid (FIG. 3D). Incubation of PEEC and authentic hepoxilin A₃ in acidified acetonitrile resulted in similar breakdown profiles—a more hydrophobic peak having a prominent mass peak of 325 and two more hydrophilic breakdown peaks having masses of 239 and 118 (FIG. 3D).

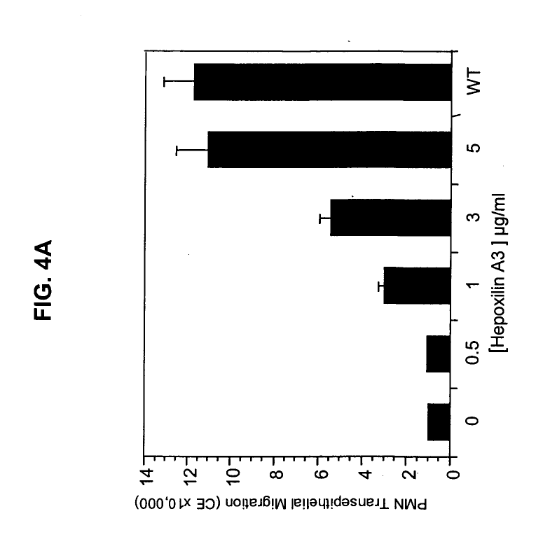

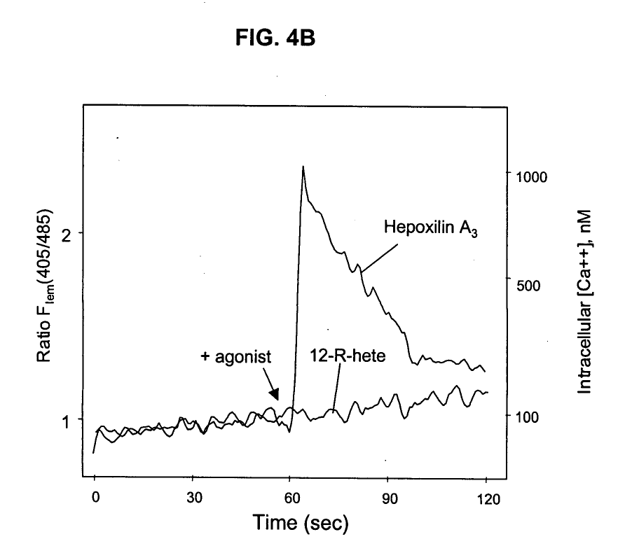

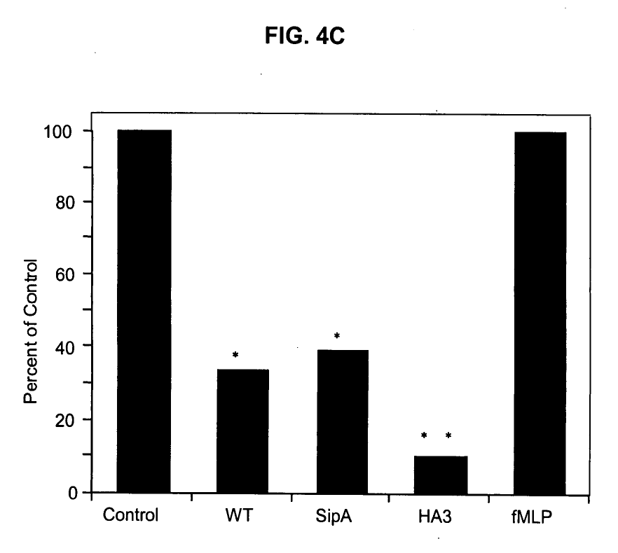

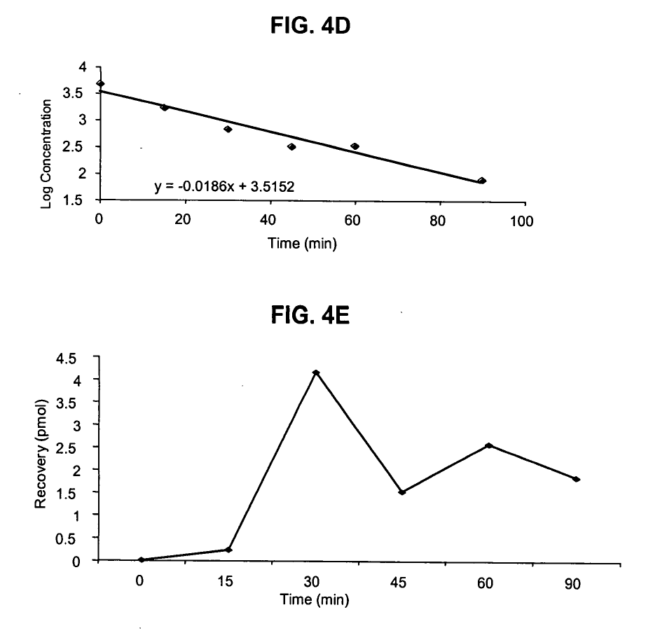

To substantiate the notion that hepoxilin A₃ was in fact the molecule responsible for PEEC bioactivity, it was examined whether synthetic hepoxilin A₃ (BIOMOL, Plymouth Meeting, Pa.) could recapitulate PEEC bioactivity. It was found that imposed gradients of synthetic hepoxilin A₃ across T84 cell monolayers induced transepithelial migration of and elicited an increase in [Ca²⁺] in PMN (FIG. 4A and 4B) that was indistinguishable from that induced by PEEC isolated following bacterial-epithelial interactions. Further, like PEEC but in contrast to chemoattractants such as IL-8, LTB₄, and formyl-methionyl-leucyl-phenylalanine (fMLP), hepoxilin A₃ did not induce PMN degranulation as assessed by elastase release (data not shown). Thus, authentic hepoxilin A₃ recapitulates all known aspects of PEEC bioactivity consistent with the notion that hepoxilin A₃ is in fact PEEC's active component.

### Example 4

### Hepoxilin A3 Stimulates Neutrophil Transmigration Across Intestinal Epithelium

It was further examined if hepoxilin A₃ acts in a gradient fashion to drive PMN migration from the basolateral to the apical epithelial surface. For these experiments, the physiologically directed (basolateral-to-apical) PMN transepithelial migration assay using cell culture inserts of inverted T84 monolayers has been used (McCormick et al., 1993; Parkos et al., 1992). Human PMN were isolated from normal volunteers, as described elsewhere (Parkos et al., 1992; Henson and Oades, 1975). PMN transmigration results were represented as PMN cell equivalents (CE) derived from a daily standard PMN dilution curve. PMN which completely traversed the monolayer were represented as the number of PMN (CE/ml in a total volume of 1 ml). Values were expressed as the mean and SD of an individual experiment done in triplicate repeated at least 3 times.

First, any potential gradient by adding excess synthetic hepoxilin A₃ to the basolateral compartment was counteracted. As shown in FIG. 4C, the addition of hepoxilin A₃ to the basolateral surface of T84 cell monolayers significantly diminished PMN transepithelial migration induced by either S. typhimurium, purified recombinant SipA, or imposed gradients of hepoxilin A₃. As expected, PMN migration to imposed gradients of fMLP was not affected by the basolateral addition of hepoxilin A₃. The establishment of a hepoxilin A₃ gradient was next measured quantitatively. As shown in 4D, hepoxilin A₃ added exogenously to either the apical or basolateral compartments of T84 monolayers resulted in the rapid loss of the molecule; in separate two experiments we observed an average 50% loss of intact hepoxilin A₃ from the apical chamber in 82 minutes (77 and 87 minutes) and in only 43 minutes (49 and 37.5 minutes) in the basolateral compartment. Hepoxilin A₃ decay in both compartments was exponential and the above stated T_{1/2} values were determined by analyzing the slope of a best-fit line to a log-linear analysis of each data set. Because of the complex degradation events occurring in both the apical and basolateral compartments, no attempt was made to calculate a transport rate. Instead, the extent of recovery of hepoxilin A₃ from the basolateral compartment following an apical application of 4500 pmoles was measured. The amount of intact hepoxilin A₃ recovered from the basolateral compartment over 90 minutes (FIG. 4E) suggested that a low but detectable amount of the molecule could reach PMNs present below the epithelial tight junction; an event essential for the establishment of a chemoattractant gradient required for PMN transmigration. Together, these results suggest that hepoxilin A₃ can establish a gradient across the epithelial tight junction, which would be required to promote PMN transepithelial migration.

### Example 5

### Liberation of PEEC/Hepoxilin A₃ Requires 12-lipoxyzenase Activity

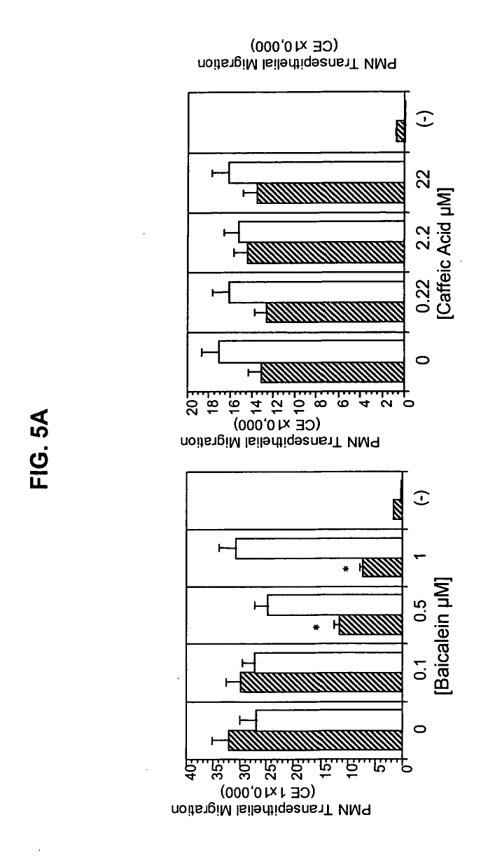

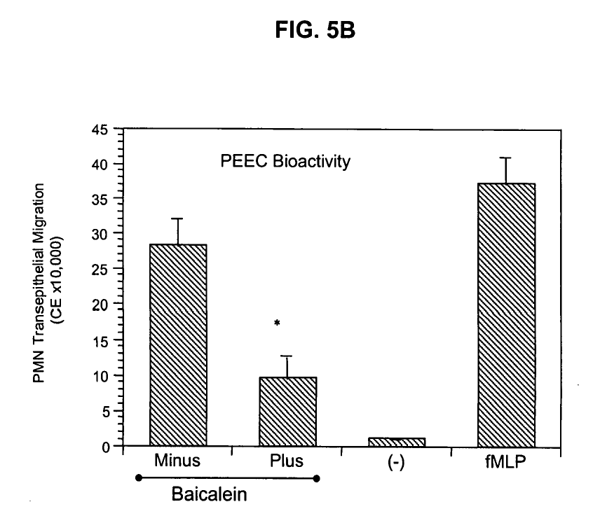

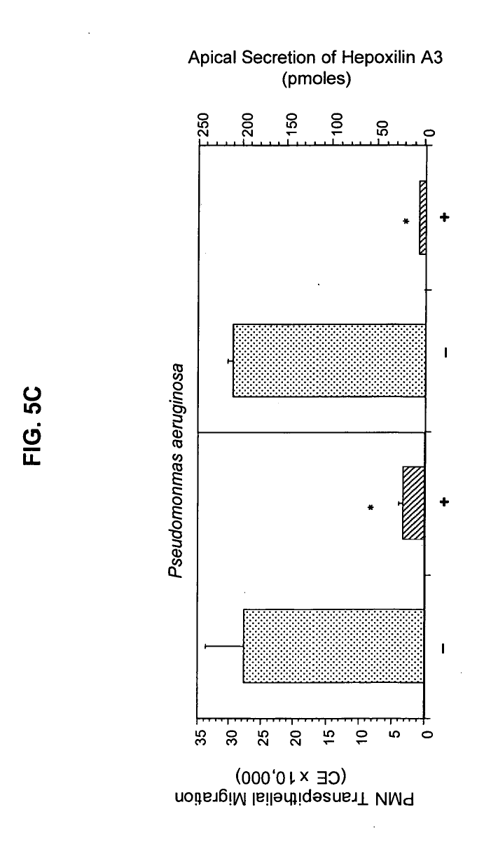

Hepoxilin A₃ is formed via the 12-lipoxygenase (LO) metabolism pathway of arachidonic acid. Therefore, the involvement of the 12-LO pathway in the ability of the enteric pathogen, S. typhimurium, to induce PMN transepithelial migration and elicit PEEC secretion was tested. Pre-treatment of T84 cells with baicalein, a 12-LO enzyme inhibitor, attenuated S. typhimurium-induced PMN transepithelial migration in a concentration-dependent fashion (FIG. 5A). For 12-LO inhibition, HBSS(+)-washed T84 cell monolayers were incubated in the presence baicalein [stock concentration at 1 mM in DMSO] for 48 hours at 37° C. Subsequently, S. typhimurium SL1344 was added to the apical surface of the T84 monolayers and after an incubation of 1 hour at 37° C., the cells were washed free of non-adherent bacteria and then processed for the PMN transmigration assay. This effect was specific to the 12-LO pathway as an inhibitor of the 5-LO pathway (responsible for LTB₄ synthesis), caffeic acid, failed to prevent S. typhimurium-induced PMN transepithelial migration (FIG. 5A). For 5-LO inhibition, the T84 cells were washed with HBSS(+) and then were incubated for 24 hours in the presence of caffeic acid (stock concentration at 22 mM in DMSO). Both inhibitors were purchased form Biomol, Plymouth Meeting, Pa.

An important control was that neither the 12-LO nor the 5-LO enzyme inhibitors prevented the secretion of IL-8 (577±35 pg/ml and 558±26 pg/ml vs. 581±31 pg/ml, respectively for S. typhimurium infected T84 cell monolayers in the presence of baicalein [1 μM] and caffeic acid 22 [μM] vs. the absence of drug treatment; 23±3 pg/ml was the value associated with control uninfected monolayers) indicating that these compounds did not non-specifically block all responses to the bacteria.

Based on this observation, it was further determined whether the activation of the 12-LO metabolic pathway was directly linked to the secretion of PEEC. T84 cell monolayers were infected with S. typhimurium in the absence or presence of the 12-LO enzyme inhibitor and apical supernatants were examined for PEEC bioactivity. As shown in FIG. 5B, treatment of T84 cell monolayers with baicalein at concentrations which significantly reduced S. typhimurium-induction of PMN transepithelial migration, showed a significant decrease in PEEC secretion. Moreover, since it had been previously shown that S. typhimurium via its effector SipA initiates an ARF6-dependent lipid-signaling cascade that directs the activation of PKC and release of PEEC, it was further examined if blocking PKC activity (which would occur upstream of 12-LO) also corresponds with a reduction in hepoxilin A₃ bioactivity and release, as one would predict. Indeed, it was found that treatment of T84 cell monolayers with 5 μM chelerethyrine chloride, a pan-PKC inhibitor, prior to infection with wild-type S. typhimurium completely ablated the ability of model intestinal epithelia to elicit PEEC/hepoxilin A3 release (57.3 pmoles and <1.0 pmole, respectively, in the absence and presence of chelerethyrine chloride).

### Example 6

### Hepoxilin A3 Stimulates Neutrophil Transmigration Across Lung Epithelium

It was further determined whether similar hepoxilin A₃ recruitment of PMN by apically attached pathogens might occur at other mucosal surfaces. Pseudomonas aeruginosa is the most prominent bacterial spp. present during chronic infection in cystic fibrosis and chronic obstructive pulmonary disease patients and is accompanied by severe inflammation, including the emigration of PMN to the airway lumen (Pizurki et al., 2000; Jahn et al., 2000; Weiss, S. 1989). As a model for the airway epithelium the A549 cell line was used. This cell line is capable of forming a polarized barrier, and is widely employed as a model for the airway epithelial surface (Smart and Casale, 1994). It was found that the addition of P. aeruginosa to the apical surface of A549 cell monolayers induced a robust PMN transepithelial migration response, which was inhibited by the addition of a 12-LO inhibitor (FIG. 5C) as well as by the disruption of the hepoxilin A₃ gradient (i.e, addition of excess hepoxilin A₃ to the basolateral compartment; data not shown). This result is consistent with the finding that treatment of the A549 cell monolayers with the12-LO inhibitor prior to infection with P. aeruginosa releases no detectable levels of hepoxilin A₃ apically as compared to untreated control monolayers (FIG. 5C). Finally, the secretion of hepoxlin A₃ in vitro was quantified using this A549 model system. For these experiments, A549 lung epithelial cells (ATTC) were grown in F12K media (Invitrogen) with 10% FBS, and maintained in culture similar to the T84 cell line. The wild type P. aeruginosa (PAO1) was used for infection of A549 after overnight aerobic growth (Luria broth) at 37° C.

The apical compartment of A549 cell monolayers treated for 90 minutes with P. aeruginosa had approximately 210 pmoles in the apical compartment and nearly 130 pmoles in the basolateral compartment. In both samples we also observed a potential hepoxilin A3 degradation peak; having a mass consistent with a di-hydroxylated species that would result from a water addition at the epoxide moiety (trioxilin A3). This potential metabolite was present at approximately 20 times greater concentration than hepoxilin A₃. Moreover, incubation with the 12-LO inhibitor resulted in no detectable levels of either hepoxilin A₃ (or the potential metabolite) in either the apical or basolateral compartments. Collectively, these data suggest that bacterial interaction with the apical surface of lung epithelial cells results in the secretion of the arachadonic acid metabolite, hepoxilin A₃, and mediates the process of transepithelial migration. Thus, activation of the signaling pathway leading to hepoxilin A₃ release may represent a novel and important component of innate immunity.

### Example 7

### Examination of Metabolic Pathways Involving Lipoxygenase Activity

Arachidonic acid is the initial substrate used by a variety of metabolic pathways involving lipoxygenase (LO) activities—enzymes that add hydroxyl groups at specific carbon atoms of this 20-carbon fatty acid. A number of these hydroxylated lipid species have been demonstrated to have a variety of biological activities related to PMNs. Several pathways, initiated by the actions of the 5-LO, 8-LO, 12-LO and 15-LO enzymes, generate arachidonic acid metabolites that have been shown to modulate PMN intracellular Ca²⁺ levels and/or to activate PMN chemotaxis. Therefore, in addition to hepoxilin A₃, a wide range of arachidonic acid metabolites for their ability to incite the transmigration of PMNs across T84 monolayers in vitro was examined (Table 1). Hepoxilin A₃ produced a saturated response at concentrations where components of the 8-LO and 15-LO pathways showed only marginal activity. Interestingly, in this assay one component of the 5-LO pathway, LTB₄, could also incite a substantial amount of PMN transmigration. This is consistent with previous reports that LTB4 is capable of inducing PMN transmigration. However, it is unlikely the LTB4 contributes to PEEC activity because LTB4 causes activation and degranulation, whereas PEEC does not, and our analytical studies suggested that PEEC is not LTB₄. Arachidonic acid, itself, added to T84 monolayers could enhance the amount of PMN transmigration, presumably through an increase in substrate availability for PEEC synthesis (Table 1). Other lipids, such as sphingolipids or linoleic acid, failed to demonstrate any PEEC activity.

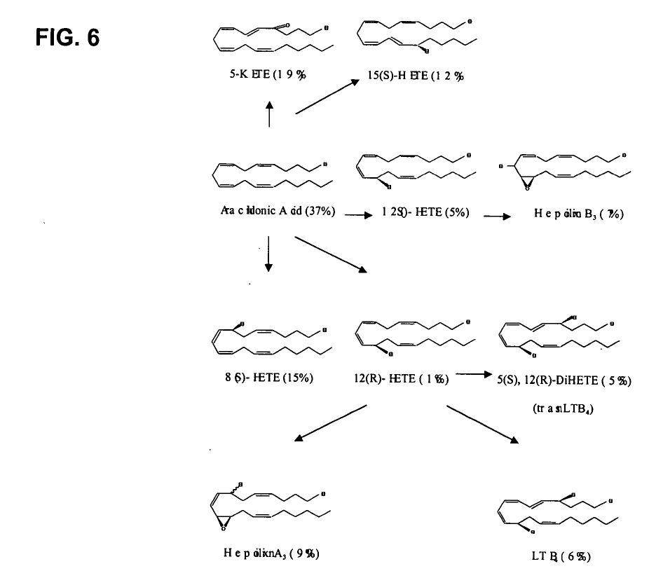

Using a variety of arachidonic acid metabolites, the structural requirements of PEEC activity were examined. Hepoxilin A₃ contains several required features for activity. The epoxide structure coupling carbons 11 and 12 is also present in the inactive molecule hepoxilin B₃, but positioned in a different chiral orientation. Hydroxylation at the 12 position was found to be insufficient since no PMN transmigration activity was observed for 12(S)-HETE or 12(R)-HETE (FIG. 6). Hepoxilin A₃ also has a hydroxyl group at the 8 carbon. The presence of this 8-OH group, however, does not appear to be sufficient for PEEC activity since 8-HETE was not capable of reconstituting PEEC activity (FIG. 6). Further, the impact of hydroxyl location at other sites on PEEC activity was explored. Hydroxylation at either the 5 carbon (trans LTB₄ or LTB₄) or the 10 carbon (hepoxilin B₃) failed to support events observed with hepoxilin A₃. Although trans LTB₄ or LTB₄ both produced outcomes consistent with their previously shown function in inflammatory events (Ford-Hutchinson, 1990), these effects appear to be correlated with PMN degranulation, something not observed with PEEC or hepoxilin A₃. Inhibitor studies to block the 5-LO pathway mirrored these outcomes obtained when specific molecules were added to an in vitro epithelial PMN transmigration assay. Further, 5-KETE, a 5-LO pathway component previously shown to stimulate eosinophil migration (Powell et al., 1995a) and mobilize PMN Ca²⁺ (Powell et al., 1995b), failed to effect a transmigration of PMNs in our system (FIG. 6). Hydroxylation at the 15 position was also not required for PEEC activity; epithelial cells incubated with a potent 15-LO inhibitor failed to block PEEC production (data not shown) and only a low activity of PMN transmigration was observed following the addition of 15(S)-HETE (FIG. 6). Collectively, data is consistent with the requirements of hepoxilin A₃ features as necessary for PEEC activity and describe the combination of unique chemical structures of this compound as critical for its observed activity as a trans-epithelial neutrophil chemoattactant.

### Example 8

### In Vivo Histopathology of Human Intestinal Xenografts Infected with S. typhimurium

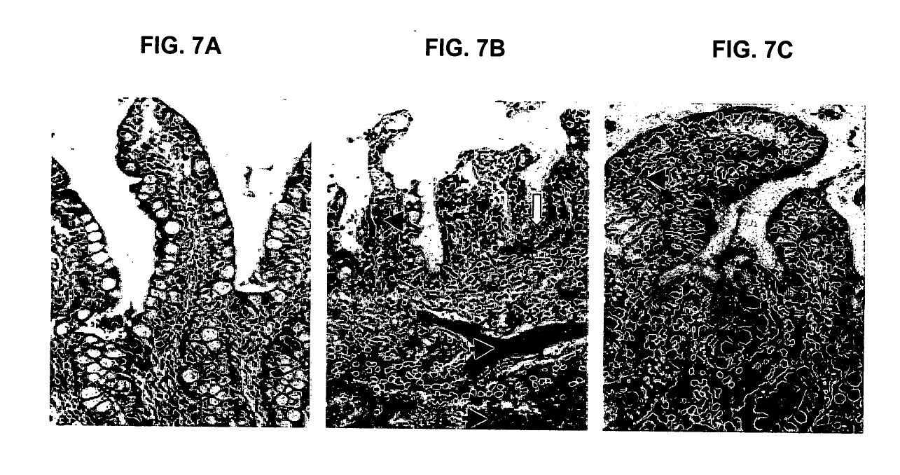

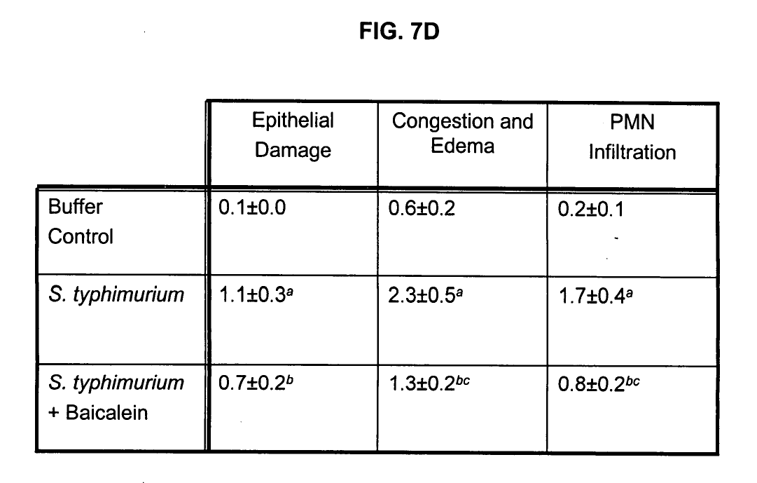

Further, the effect of baicalein on S. typhimurium pathology in human intestinal xenografts was determined (Savidge et al., 1995) (FIG. 7). The human fetal intestinal xenograft model used in the present study has been previously described in detail (Savidge et al., 1995). Briefly, human fetal small intestine (n=3, gestational age 10-14weeks) was transplanted subcutaneously into C.B-17 severe-combined immunodeficient (SCID) mice. Xenografts were allowed to develop for a period between 10-20 weeks before use at which time the epithelium and underlying mucosa is fully differentiated (Savidge et al., 1995). Xenografts were infected with approximately 5×10⁷ wild type S. typhimurium SL1344 in sterile HBSS(+) buffer in a 100 μl volume injected intralumenally by subcutaneous injection. Xenografts receiving the drug treatment were injected with 1 μM baicalein in a 100 μl volume 2 hours prior to infection with S. typhimurium (in the continued presence of 1 μM baicalein). Xenograft tissue was removed 15 hours after infection, extensively washed, and snap frozen in OCT compound.

Xenografts that were infected with wild type S. typhimurium exhibited a severe pathology compared to control (buffer treated) injected xenografts (FIG. 7A and 7B). The histopathology associated with the S. typhimurium-infected xenografts demonstrated profuse dissemination of red blood cells in the mucosa and sub-mucosa with a noticeable PMN infiltrate. These xenografts showed evidence of crypt abscesses, crypt hyperplasia, and villus tip atrophy. A severe vascular disturbance was also noted. These outcomes are all consistent with the clinical manifestations observed from S. typhimurium infection in humans. By contrast, the xenografts infected with pathogenic S. typhimurium in the presence of the 12-LO inhibitor (baicalein) displayed a different response, with only minimal alterations detectable (FIG. 7C). As shown in FIG. 7D, histological evaluation of the intestinal inflammation was graded by a score of 0-3 for epithelial damage, vascular congestion and edema of the mucosa, and PMN margination and infiltration as previously described (Triadafilopoulos et al., 1987). The results demonstrate that the severity intestinal inflammation induced by S. typhimurium, measured by three separate criteria, were significantly reduced when treated in the presence of the 12-LO inhibitor, baicalein. Importantly, interruption of the 12-LO synthetic pathway (required for the production of hepoxilin A₃) was shown to impede the transmigration of PMNs in vivo.

It was further confirmed that treatment of the intestinal xenografts with baicalein inhibited the 12-LO pathway by measuring the amount of 12-S-HETE, a major 12-LO metabolite, released into the lumenal fluid. Using an established 12-S-HETE ELISA assay (Assay Designs, Ann Arbor, Mich.) it was determined that lumenal fluid collected from S. typhimurium-infected xenografts in the absence of baicalein treatment contained 114.5 ng/ml 12-S-HETE, whereas the lumenal fluid collected from S. typhimurium-infected xenografts in the presence of baicalein treatment contained 21.5 ng/ml 12-S-HETE (average amounts from two separate intestinal lumens). Likewise, it was found that the apical supernatants of S. typhimurium infected T84 cell monolayers contained 12-S-HETE (525 pg/ml vs. 104 pg/ml, respectively for S. typhimurium infected vs. the uninfected control), and that secretion of 12-S-HETE during S. typhimurium infection was restricted to the apical surface (573 pg/ml vs 113 pg/ml compared to 117 pg/ml, respectively for the apical vs. basolateral compartment compared to the uninfected control; all values represent average amounts from two separate experiments). Finally, it was determined that there was no statistical difference in the amount IL-8 produced in the S. typhimurium-infected intestinal xenografts in the absence compared to the presence of baicalein treatment (200±53 pg/mg compared to 225±30 pg/mg, respectively, verses 52±11 pg/mg for uninfected control). Taken together, these in vivo observations indicate that the 12-LO inhibitor, baicalein, inhibits metabolites of the 12-LO pathway but has no effect on IL-8 production, and thus are consistent with the in vitro results.

### Example 9

### Treatment of Intestinal Inflammation with Baicalein In Vivo

The therapeutic benefits of baicalein on subsiding intestinal inflammation, which was induced in SCID mice by i.v. injection of CD4+ CD45RB HI T cells, was examined. For these experiments, spleens were collected from 6-12 week old donor mice (BALB/c) and splenocytes were isolated by mechanical homogenization of whole spleens. The CD4+ T cells were selected by positive selection. In brief, a cell suspension of pooled splenocytes from 4-5 donor mice was incubated with anti-CD4 (L3T4) antibody coated magnetic beads (DYNABEADS: Catalog # 114.05, Dynal, Lake Success, N.Y.) for 20-30 minutes at 4° C. and separated by magnetic cell sorting with a Dynal Magnetic Particle Concentrator (MPC). Cells were removed from the cell-bead complex with Dynal DETACHaBEAD, and isolated from beads using a Dynal MPC. The resulting CD4+ enriched population was >90% pure. The cell suspension was then incubated with Fc block (anti-CD32, PharMingen, 01241A) and labeled with anti-CD4-FITC (PharMingen, 9004D) and anti-CD45RB-PE (PharMingen, 01145A), for 30 minutes at 4° C. washed, and sorted using a FACSTAR (Becton Dickinson, San Jose, Calif.) cell sorter. Double positive cells (CD4+/CD45Rb+) were collected, selecting the cells that expressed high levels of CD45Rb (brightest 45%). The collected cell population was >90% pure and viable. Cells were then washed in cold potassium buffered saline (PBS Sigma D8662) and resuspended in PBS. Balb/c SCID mice, aged 6-12 weeks, were injected intravenously with CD45RBHI Tcells in the tail vein.

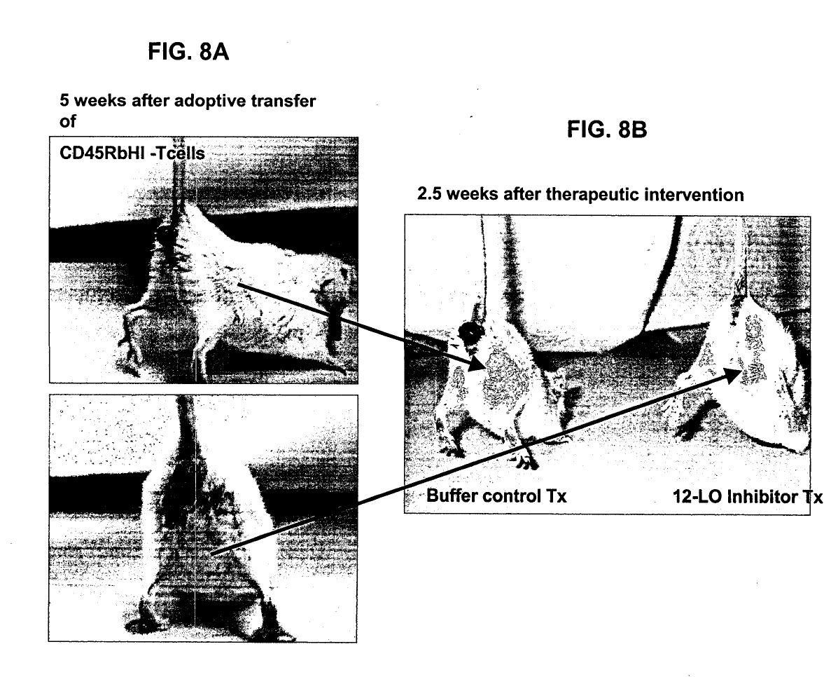

Ten weeks post CD45RBHI T cell transfer, roughly 30% of SCID mice developed intestinal inflammation, characterized by mucin depletion, goblet cell loss, epithelial hyperplasia and finally rectal prolapse (FIG. 8). These mice were divided equally among treatment groups The first treatment group received buffer control alone (sterile Hanks Balanced Salts Solution (“HBSS”)) and the second and the third treatment groups received low (2 μM) and high (200 μM) doses of baicalein i.v., respectively, every other day along with a vehicle control.

As shown in FIG. 8B, diseased SCID mice which received the buffer control alone or low doses of baicalein (2 μM) for 2.5 weeks, showed no improvement of their health status and rectal prolapse. In contrast, diseased SCID mice which received high doses of baicalein (200 μM), showed complete recovery of rectal prolapse. The results of these experiments demonstrate that intestinal inflammation in SCID mice, which is characterized by, for example, rectal prolapse, was significantly reduced when treated in the presence of the 12-LO inhibitor, baicalein.

### Example 10

### Hepoxilin A3 Transport

A transport mechanism mediating hepoxilin A₃ secretion was identified as described herein. It is now known that Hepoxilin A₃ which is a negatively-charged lipophilic molecule, can be transported by the P-glycoprotein class and/or multidrug resistance class of proteins present in the apical plasma membrane of human intestinal epithelial cells.

Due to the lipophilic nature of hepoxilin A3, it was hypothesized that this molecule might be released from intestinal epithelial cells by ATP-binding cassette transporters. These transporters were initially considered because substrates previously identified for these proteins include hydrophobic compounds of the molecular weight range of hepoxilin A3 (Borst et al. (1999) Biochimica et Biophysica Acta, 1461:347-357; Borst et al. (2000) J.Natl.Cancer Inst. 92:1295-1305; Leier et al. (1994) J. Biol. Chem. 269: 27807-27810).

To test the involvement of ATP-binding cassette transporters on the hepoxilin A3 secretion, specific MDR inhibitors, such as, cyclosporine A, verapamil, ethacrynic acid and probenecid, were applied to T84 cell monolayers and monitored for the ability to induce PMN transepithelialmigration in the presence of S. typhimurium. In each instance, the T84 cell monolayers were incubated for 1 hour in the presence of the inhibitor prior to the addition of S. typhimurium. In all cases, the drugs (at the doses used) neither altered the ability of bacteria to adhere or enter into the T84 cells nor did these drugs alter barrier function or permeability.

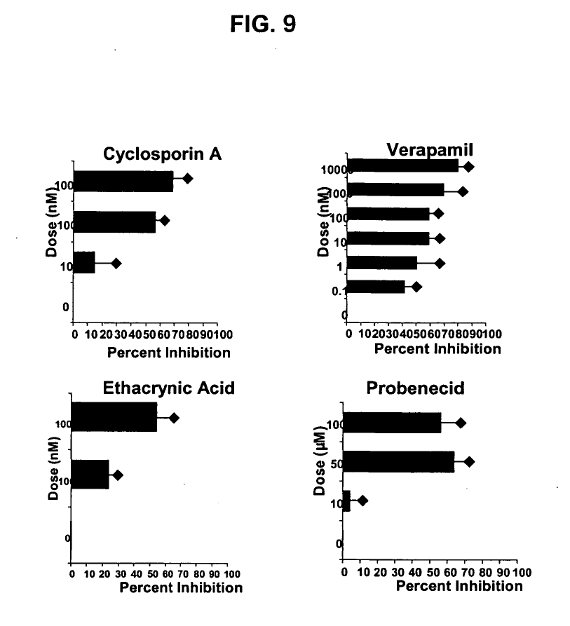

As shown in FIG. 9, cyclosporine A and verapamil, the first generation modulators of P-glycoproteins, produced a marked (>70%) inhibition of the S. typhimurium-induced PMN transepithelial migration. Further, both ethacrynic acid, which had been shown to inhibit glutathione S-transferase and efflux of LTC4 via the MRP-2 transporter, and probenecid, which had been shown to clog the MRP-2 transporter and thus prevent the efflux of substrates which use this transporter, significantly inhibited the ability of S. typhimurium to induce PMN transepithelial migration. This assertion is supported by pharmacological analysis (FIG. 9) which shows that 80 nM cyclosporine A as well as 50 μM probenecid can inhibit more than 50% of the hepoxilin A₃ secreted in a pathogen-induced fashion from T84 cell monolayers (˜0.5 nM vinblastine also inhibited nearly 60% of the hepoxilin A3 secreted). The results of these experiments suggest that hepoxilin A3 is secreted from the apical plasma membrane of intestinal epithelia cells via the MRP-2 isoform of this protein family. Such studies, however, do not rule out the potential involvement of other transporters for hepoxilin A3.

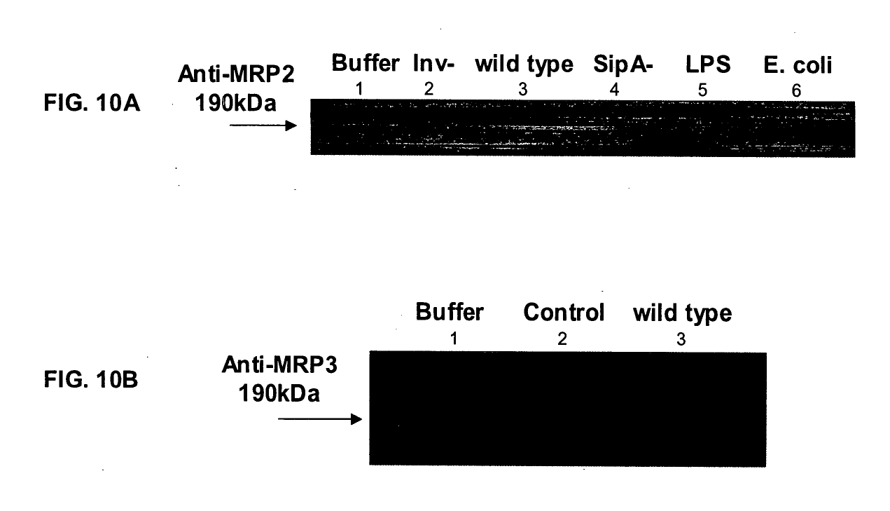

The specificity of Hepoxilin A3 secretion from the apical plasma membrane of intestinal epithelial cells via the MRP-2 isoform of the MRP family was investigated. For these experiments, T84 cell were infected with wild type S. typhimurium (FIG. 10A, lane 3), or an invasion defective strain (FIG. 10A, lane 2), a SipA mutant strain (FIG. 10A, lane 4), LPS (FIG. 10A, lane 5), or a commensal E. coli strain (FIG. 10A, lane 6). An uninfected buffer treatment (FIG. 10A, lane 1) was used as the negative control. T84 cells were lysed and immunprecipitated with monoclonal antibodies specific for MRP-2. Immune complexes were recovered with protein sepharose-G slurry. Immunoprecipitates were washed, electrophoresed, and Western blotted. Only wild type S. typhimurium induced the up-regulation of MRP-2 on T84 cells.

To further show specificity for this efflux pathway, immunoprecipitation experiments with antibodies specific for MRP-3 were performed. As expected, wild type S. typhimurium failed to promote the up-regulation of this transporter (FIG. 10B).

Of the six members comprising the MRP family (MRP1-6), only MRP-2 is routed to the apical surface of polarized epithelial cells (Borst et al. (1999) Biochimica et Biophysica Acta, 1461:347-357; Borst et al. (2000) J.Natl.Cancer Inst. 92:1295-1305). The most abundant constitutive expression of human and rat MRP-2/MRP-2 mRNA was found in the renal proximal tubule brush-border membrane and the hepatocyte canalicular membrane. Lower levels of MRP-2 were observed in the small intestine, exclusively localized to the apical brush border membrane of vili. Importantly, MRP-2 expression decreases in intensity from the villus tip to the crypts, where no expression was observed. Other parts of the duodenum, such as the submucosa and muscle layers are known to be negative for MRP-2 expression (Van Aubel et al. (2000) European J. Pharmacology, 400:195-198).

Thus, the marked increase in MRP-2 expression in the crypt regions of inflamed tissue, which was in contrast to the basal MRP-2 expression in normal intestinal tissue, was anticipated. Given this conclusion, it was also presumed that in the efflux pathway for hepoxilin A₃, MRP-2, would be activated during the disease phase and suppressed upon baicalein treatment.

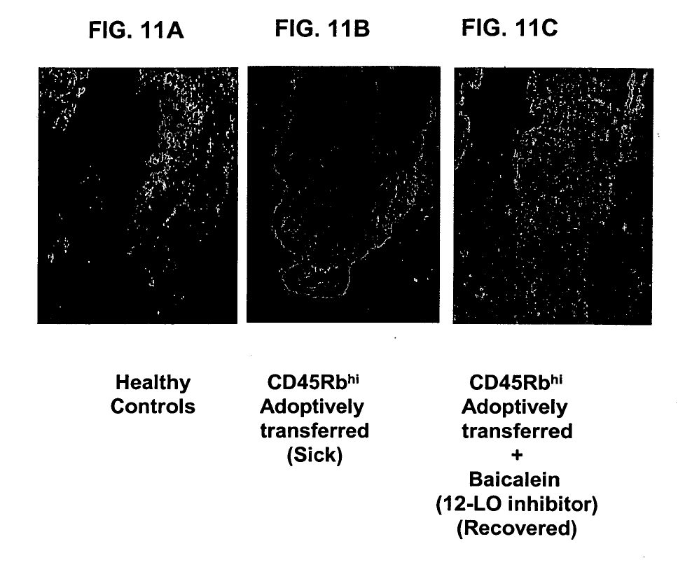

To test this hypothesis, mice were adoptively transferred with the CD45Rbhi subset of T cells and after six weeks the animals started to exhibits signs of illness (rectal prolapse, diarrhea, weight loss). The sick mice that were treated with a therapeutic regiment of baicalein, the 12-LO inhibitor, resolved the illness after 2 weeks. The upper third of the large bowel from these mice were excised, and fixed in OCT. Slides from these tissues were prepared and fluorescently probed for MRP-2 expression. As shown in FIG. 11A, the healthy controls do not express MRP-2, where as the sick mice exhibited a profound expression pattern of MRP-2 at the apical surface of the intestinal epithelium (FIG. 11B). Interestingly, following treatment with baicalein such expression of MRP-2 was completely suppressed (FIG. 11C). Thus, in the efflux pathway for hepoxilin A3, MRP-2 is in fact activated during the disease phase and suppressed upon baicalein treatment

Thus, at least one essential participant in the apical secretion of hepoxilin A₃ (MRP-2) has been determined, providing a new target for inhibiting hepoxilin A3 activity.

### References

Boulianne, G. l., Hozumi, N., and Shulman, M. J. (1984) Production of functional chimeric mouse/human antibody. Nature 312(5995):643-6.

Dharmsathaphorn, K., and Madara, J. L. (1990) Established intestinal cell lines as model systems for electrolyte transport studies. Methods Enzymol. 192: 354-389.

Ford-Hutchinson, A. W. (1990) Leukotriene B4 in inflammation. Crit. Rev. Immunol. 10: 1-12.

Gewirtz, A. T., Fokin, V. V., Petasis, N. A., Serhan, C. N., Madara, J. L. (1999) LXA4, aspirin-triggered 15-epi-LXA4, and their analogs selectively downregulate PMN azurophilic degranulation. Am J Physiol. 276: C988-C994.

Gewirtz, A. T., Siber, A. M., Madara, J. L., McCormick, B. A. (1999) Orchestration of neutrophil movement by intestinal epithelial cells in response to Salmonella typhimurium can be uncoupled from bacterial internalization. Infect. Immun. 67: 608-617.

Gewirtz, A. T., Simon, P. O., Schmitt, C. K., Taylor, L. T., Hagedorn, C. H., O'Brien, A. D., Neish, A. S., Madara, J. L. (2001) Salmonella typhimurium translocate flagellin across intestinal epithelia, inducing a proinflammatory response. J. Clin. Invest. 107: 27-30. Hammerling (1981). Monoclonal Antibodies and T-cell Hybridomas, Elsevier, N.Y. pp.563-681.

Henson, P., and Oades Z. G. (1975) Stimulation of human neutrophils by soluble and insoluble immunoglobulin aggregates. J. Clin. Invest. 56: 1053-1061.

Hueck, C. J., Hantman, M. J., Bajaj, V., Johston, C., Lee, C. A., Miller, S. I. (1995) Salmonella typhimurium secreted invasion determinants are homologous to Shigella Ipa proteins. Mol. Microbiol. 16: 855-864.

Imler, J. L., and Hoffman, J A. (2000) Toll receptors in innate immunity. Trends Cell Biol 11: 304-311.

Jahn, H. U., Krull, M., Wuppermann, F. N., Klucken, A. C., Rosseau, S., Seybold, J., Hegemann, J. H., Jantos, C. A., and Suttorp, N. (2000) Infection and activation of airway epithelial cells by Chlamydia. J. Infect. Dis. 182: 1678-1687.

Kluskens, L., Lee, W., and Kohler, H.(1976) Immune response to phosphorylcholine. Characterization of the epitope-specificity. Eur J Immunol. 5(7):489-96.

Kohler, G., and Milstein, C. (1975) Continuous cultures of fused cells secreting antibody of predefined specificity. Nature 256(5517):495-7.

Kumar, N. B., Nostrant, T. T., Appelman, H. D. (1982) The histopathologic spectrum of acute self-limited colitis (acute infectious type colitis). Am. J. Surg. Path. 6: 523-529.

Lam, K. S. (1991) A new type of synthetic peptide library for identifying ligand-binding activity, Nature 354:82-84.

Lee, C. A., and Falkow, S. (1990) The ability of Salmonella to enter mammalian cells is affected by bacterial growth state. Proc. Natl. Acad. Sci. USA 87: 4304-4308.

Lee, C. A., Silva, M., Siber, A. M., Kelly, A. J., Galyov, E., McCormick, B. A. (2000) A secreted Salmonella protein induces a proinflammatory response in epithelial cells which promotes neutrophil migration. Proc. Natl. Acad. Sci. USA. 97:12283-12288.

Madara, J. L., Parkos, C. A., Colgan, S. P., MacLeod, R. J., Nash, S. Matthews, J., Delp, C., Lencer, W. (1992) C1-secretion in a model intestinal epithelium induced by a neutrophil-derived secretagogue. J. Clin. Invest. 89: 1938-1944.

McCormick, B. A., Colgan, S. P., Archer, C. D., Miller, S. I., Madara, J. L. (1993) Salmonella typhimurium attachment to human intestinal epithelial monolayers: transcellular signaling to subepithelial neutrophils. J. Cell. Biol. 123: 895-907.

McCormick, B. A., Parkos, C. A., Colgan, S. P., Carnes, D. K., Madara, J. L. (1998) Apical secretion of a pathogen-elicited epithelial chemoattractant (PEEC) activity in response to surface colonization of intestinal epithelia by Salmonella typhimurium. J. Immunol. 160 455-466.

McCormick, B., Hofman, P., Kim, J., Carnes, D., Miller, S. I., Madara, J. L. (1995) Surface attachment of Salmonella typhimurium to intestinal epithelia imprints the subepithelial matrix with gradients chemotactic for neutrophils. J. Cell. Biol. 131: 1599-1608.

McGovern, V. J. and Slavutin, L. J. (1979) Pathology of Salmonella colitis. Am. J. Surg. Pathol. 3: 483-490.

Morrison D. C. (1985) Transfectomas provide novel chimeric antibodies. Science 229(4719): 1202-7.

Nash, S., Parkos, C. A., Nusrat, A., Delp, C., Madara, J. L. (1991) In vitro model of intestinal crypt abscess: a novel neutrophil-derived secretagogue activity. J. Clin. Invest. 87: 1474-1477.

Neuberger, M. S., Williams, G. T., Mitchell, E. B., Jouhal, S. S., Flanagan, J. G., Rabbitts, T. H. (1985) A hapten-specific chimeric IgE antibody with human physiological effector function. Nature 314 (6008): 268-70.

Nusrat, A., Turner, J. R., Madara, J. L. (2000) Molecular physiology and pathophysiology of tight junctions. IV. Regulation of tight junctions by extracellular stimuli, nutrients, cytokines, and immune cells. Am. J. Physiol. Gastrointest. Liver Physiol. 279: G851-G858.

Pace-Asciak, C. R., Reynaud, D., Demin, P., Nigam, S. (1999) The hepoxilins. A review. Adv. Exp. Med. Biol. 447:123-133.

Parkos, C. A., Colgan, S. P., Delp, C., Arnaout, M. A., Madara, J. L. (1992) Neutrophil migration across a cultured epithelial monolayer elicits a biphasic resistance response representing sequential effects on transcellular and paracellular pathways. J. Cell. Biol. 117: 757-764.

Pizurki, L., Morris, M. A., Chanson, M., Solomon, M., Pavirani, A., Bouchardy, I., Suter, S. (2000) CFTR does not affect neutrophil migration across cystic fibrosis airway epithelial monolayers. Am J. Pathol. 1556: 1407-1416.

Petersdorf, R. G. (1983) Harrison's Principle of Internal Medicine, 10^{th} Edition, McGraw-Hill, NY, N.Y.

Physician's Desk Reference (1987) 41^{st} ed., Medical Economics Co., Inc. 1103-1104.

Powell, W. S., Chung, D., Gravel, S. (1995a) 5-oxo-6,8,11,14-eicosatetraenoic acid is a potent stimulator of the human eosinophil migration. J. Immunol. 154: 4123-4132.

Powell, W. S., Gravel, S., Gravelle, F. (1995b) Formation of 5-0×0-metabolite of 5,8,11,14,17-eicosa pentaenoic acid and its effects on human neutrophils and eosinophils. J. Lipid Res. 36: 2590-2598.

Rout, W. R., Formal, S. B., Dammin, S. B., Giannella, R. A. (1974) Pathophysiology of Salmonella diarrhea in the Rhesus monkey: Intestinal transport, morphological and bacteriological studies. Gastroenterology. 67: 59-70.

Savidge, T. C., Morey, A. L., Ferguson, D. J. P., Fleming, K . A., Shmakov, A. N., Phillips, A. D. (1995) Human intestinal development in a severe-combined immunodeficient xenograft model. Differentiation 58: 361-371.

Songyang, Z., Shoelson, S. E., Chaudhuri, M., Gish, G., Pawson, T., Haser, W. G., King, F., Roberts, T., Ratnofsky, S., Lechleider, R. J. (1993). Cell, 72(5): 767-78.

Sutherland, M., Schewe, T., and Nigam, S. (2000). Biological actions of the free acid of hepoxilin A3 on human neutrophils. Biochem Pharmacol. 59(4): 435-40.

Smart, S. J., and Casale, T. B. (1994) Pulmonary epithelial cells facilitate TNF-alpha-induced neutrophil chemotaxis: a role for cytokine networking. J. Immunol. 152: 4087-4094.

Triadafilopoulos, G., Pothoulakis, C., O'Brien, M., and Lamont, J. T. (1987) Differential effects of Closteridium difficile toxins A and B on rabbit epithelium. Gastroenterology 93: 273-279.

Wand, J. R., and Zurawski, V. R. (1981) High affinity monoclonal antibodies to hepatitis B surface antigen produced by somatic cell hybrids. Gastroenterology, 80(2):225-32.

Wahl. R. L., Parker, C. W., and Philpott, G. W. (1983). Improved radioimaging and tumor localization with monoclonal F (ab) 2. J Nucl Med. 24 (4): 316-25.

Weiss, S. (1989) Tissue destruction by neutrophils. N. Engl. J. Med. 320: 365-376.

We claim:

1. A method of enhancing an immune response in a subject in need thereof comprising administering to the subject a pharmaceutical composition comprising hepoxilin A3 in an amount sufficient to induce inflammatory cell transmigration across an epithelial surface in a basolateral-to-apical direction.

2. The method of claim 1, wherein the subject is a human.

3. The method of claim 1, wherein the immune response is directed against a pathogenic infection.

4. The method of claim 3, wherein the pathogenic infection is in the gastrointestinal system.

5. The method of claim 4, wherein the pathogen causing the infection is Helicobacter pylori.

6. The method of claim 4, wherein the pathogen causing the infection is Salmonella typhimurium.

7. The method of claim 3, wherein the pathogenic infection is in the respiratory system.

8. The method of claim 7, wherein the pathogen causing the infection is Pseudomonas aeruginosa.

9. The method of claim 3, wherein the pathogenic infection is in the skin.

10. The method of claim 1, wherein the inflammatory cells are selected from the group consisting of monocytes, lymphocytes, eosinophils, neutrophils and basophils and combinations thereof.

11. The method of claim 10, wherein the neutrophils are polymorphonuclear leukocytes.

12. A method of enhancing an immune response in a subject in need thereof comprising administering to the subject a pharmaceutical composition comprising a hepoxilin A3 analog, wherein the analog has at least native hepoxilin A3 activity, in an amount sufficient to induce inflammatory cell transmigration across an epithelial surface in a basolateral-to-apical direction.

13. The method of claim 12, wherein the subject is a human.

14. The method of claim 12, wherein the immune response is directed against a pathogenic infection.

15. The method of claim 14, wherein the pathogenic infection is in the gastrointestinal system.

16. The method of claim 15, wherein the pathogen causing the infection is Helicobacter pylori.

17. The method of claim 14, wherein the pathogen causing the infection is Salmonella typhimurium.

18. The method of claim 14, wherein the pathogenic infection is in the respiratory system.

19. The method of claim 18, wherein the pathogen causing the infection is Pseudomonas aeruginosa.

20. The method of claim 14, wherein the pathogenic infection is in the skin.

21. The method of claim 14, wherein the analog has activity that is superior to hepoxilin A3.

22. The method of claim 12, wherein the inflammatory cells are selected from the group consisting of monocytes, lymphocytes, eosinophils, neutrophils and basophils and combinations thereof.

23. The method of claim 22, wherein the neutrophils are polymorphonuclear leukocytes.

24. A method of reducing inflammation in a subject in need thereof comprising administering to the subject a pharmaceutical composition comprising an inhibitor of hepoxilin A3 synthesis in an amount sufficient to reduce inflammatory cell migration.

25. The method of claim 24, wherein the inhibitor is a 12-lipoxygenase inhibitor.

26. The method of claim 25, wherein the inhibitor is biacalein.

27. The method of claim 24, wherein the subject is a human.

28. The method of claim 24, wherein the inflammatory cells are selected from the group consisting of monocytes, lymphocytes, eosinophils, neutrophils and basophils and combinations thereof.

29. The method of claim 28, wherein the neutrophils are polymorphonuclear leukocytes.

30. A method of reducing inflammation in a subject in need thereof comprising administering to the subject a pharmaceutical composition comprising an inhibitor of hepoxilin A3 activity in an amount sufficient to reduce inflammatory cell migration.

31. The method of claim 30, wherein the inhibitor is a 12-lipoxygenase inhibitor.

32. The method of claim 31, wherein the inhibitor is biacalein.

33. The method of claim 30 wherein the subject is a human.

34. The method of claim 30, wherein the inflammatory cells are selected from the group consisting of monocytes, lymphocytes, eosinophils, neutrophils and basophils and combinations thereof.

35. The method of claim 34, wherein the neutrophils are polymorphonuclear leukocytes.

36. A method of treating inflammation in a subject in need thereof comprising administering to the subject a pharmaceutical composition comprising an inhibitor of hepoxilin A3 synthesis in an amount sufficient to reduce inflammatory cell accumulation.

37. The method of claim 36, wherein the inhibitor is a 12-lipoxygenase inhibitor.

38. The method of claim 37, wherein the inhibitor is biacalein.

39. The method of claim 36, wherein the subject is a human.

40. The method of claim 36, wherein the inflammatory cells are selected from the group consisting of monocytes, lymphocytes, eosinophils, neutrophils and basophils and combinations thereof.

41. The method of claim 40, wherein the neutrophils are polymorphonuclear leukocytes.

42. The method of claim 36, wherein the inflammation in the subject is caused by a disorder selected from the group consisting of enterocolitis, viral infection, ulcerative colitis, diverticulitis, bacterial infection, protozoan infection, cystic fibrosis, chronic obstructive pulmonary disease, appendicitis, gastritis, Barrett's esophagus, pneumonitis; cervicitis, chronic interstitial nephritis, colitis, colonic diverticulitis, conjunctivitis, contact dermatitis, ulcer, cystitis, gingivitis, mastitis, esophagitis, pancreatitis, glomerulonephritis, inflammatory bowel disease, Crohn's disease, Addison's disease, and psoriasis.

43. A method of treating inflammation in a subject in need thereof comprising administering to the subject a pharmaceutical composition comprising an inhibitor of hepoxilin A3 activity in an amount sufficient to reduce inflammatory cell accumulation.

44. The method of claim 43, wherein the inhibitor is a 12-lipoxygenase inhibitor.

45. The method of claim 44, wherein the inhibitor is biacalein.

46. The method in claim 43, wherein the subject is a human.

47. The method of claim 43, wherein the inflammatory cells are selected from the group consisting of monocytes, lymphocytes, eosinophils, neutrophils and basophils and combinations thereof.

48. The method of claim 47, wherein the neutrophils are polymorphonuclear leukocytes.

49. The method of claim 43, wherein the inflammation in the subject is caused by a disorder selected from the group consisting of enterocolitis, viral infection, ulcerative colitis, diverticulitis, bacterial infection, protozoan infection, cystic fibrosis, chronic obstructive pulmonary disease, appendicitis, gastritis, Barrett's esophagus, pneumonitis; cervicitis, chronic interstitial nephritis, colitis, colonic diverticulitis, conjunctivitis, contact dermatitis, ulcer, cystitis, gingivitis, mastitis, esophagitis, pancreatitis, glomerulonephritis, inflammatory bowel disease, Crohn's disease, Addison's disease, and psoriasis.

50. A method of inducing inflammatory cell movement across an epithelial surface in a basolateral-to-apical direction comprising localizing hepoxilin A3 or a hepoxilin A3 analog, wherein the analog has at least native hepoxilin A3 activity, proximal to the surface in an amount sufficient to induce inflammatory cell transmigration.

51. The method of claim 30, wherein the epithelial surface is present within a tissue selected from the group consisting of intestine, kidney, stomach, liver, thyroid, trachea, lung, gall bladder, urinary bladder, bile ducts, pancreatic ducts, liver, testicles, uterus and skin.

52. The method of claim 30, wherein the inflammatory cells are selected from the group consisting of monocytes, lymphocytes, eosinophils, neutrophils and basophils and combinations thereof.

53. The method of claim 32, wherein the neutrophils are polymorphonuclear leukocytes.

54. A method of blocking inflammatory cell movement across an epithelial surface in a basolateral-to-apical direction comprising localizing an inhibitor of hepoxilin A3 synthesis or activity proximal to the surface in an amount sufficient to reduce inflammatory cell transmigration.

55. The method of claim 30, wherein the epithelial surface is present within a tissue selected from the group consisting of intestine, kidney, stomach, liver, thyroid, trachea, lung, gall bladder, urinary bladder, bile ducts, pancreatic ducts, liver, testicles, uterus and skin.

56. The method of claim 30, wherein the inflammatory cells are selected from the group consisting of monocytes, lymphocytes, eosinophils, neutrophils and basophils and combinations thereof.

57. The method of claim 36, wherein the neutrophils are polymorphonuclear leukocytes.

58. A method of screening compositions for hepoxilin A3 activity comprising the steps of:
a) contacting control cells that do not contain hepoxilin A3 with hepoxilin A3;
b) contacting test cells that do not contain hepoxilin A3 with an amount of test compound sufficient to exert a physiologic effect comprising inducing transmigration of inflammatory cells;
c) separately measuring the physiologic effect of the control cells and test cells; and
d) comparing the physiologic effect of the test compound to the physiologic effect of hepoxilin A3, wherein determination of a physiologic effect of the test compound is expressed relative to that of the hepoxilin A3.

59. The method of claim 38, wherein the test compound is a hepoxilin A3 analog.

60. The method of claim 38, wherein the control cells and test cells comprise polarized T84 cell monolayers.

61. The method of claim 38, wherein the control cells and test cells comprise polarized A549 cell monolayers.

62. The method of claim 38, wherein the inflammatory cells are selected from the group consisting of monocytes, lymphocytes, eosinophils, neutrophils and basophils and combinations thereof.

63. The method of claim 42, wherein the neutrophils are polymorphonuclear leukocytes.

64. A method of screening compositions for hepoxilin A3 inhibitory activity comprising the steps of:
a) contacting control cells that do not contain hepoxilin A3 with hepoxilin A3 and measuring the physiologic effect exhibited by the control cells;
b) contacting test cells that contain hepoxilin A3 with an amount of test compound sufficient to produce a physiologic effect whereby migration of inflammatory cells is inhibited, and measuring the physiologic effect exhibited by the test cells; and
c) comparing the physiologic effect of the test compound to the physiologic effect of hepoxilin A3, wherein determination of a physiologic effect of the test compound is expressed relative to that of the hepoxilin A3.

65. The method of claim 44, wherein the test compound is an inhibitor of hepoxilin A3 acitvity.

66. The method of claim 44, wherein the test compound is an inhibitor of hepoxilin A3 synthesis.

67. The method of claim 44, wherein the control cells and test cells comprise polarized T84 cell monolayers.

68. The method of claim 44, wherein the control cells and test cells comprise polarized A549 cell monolayers.

69. The method of claim 44, wherein the inflammatory cells are selected from the group consisting of monocytes, lymphocytes, eosinophils, neutrophils and basophils and combinations thereof.

70. The method of claim 44, wherein the neutrophils are polymorphonuclear leukocytes.

71. A pharmaceutical composition comprising hepoxilin A3, or an analog thereof having native hepoxilin A3 activity.

72. A method of reducing inflammation in a subject in need thereof comprising administering to the subject a pharmaceutical composition comprising an inhibitor of hepoxilin A3 secretion in an amount sufficient to reduce inflammatory cell migration.

73. The method of claim 72, wherein the inhibitor reduces the activity of a hepoxilin A3 transporter.

74. The method of claim 73, wherein the transporter is a MDR protein pump.

75. The method of claim 74, wherein the inhibitor is cyclosporine A.

76. The method of claim 74, wherein the inhibitor is verapamil.

77. The method of claim 74, wherein the inhibitor is ethacrynic acid.

78. The method of claim 74, wherein the inhibitor is probenecid.

79. The method of claim 72 wherein the subject is a human.

80. The method of claim 72, wherein the inflammatory cells are selected from the group consisting of monocytes, lymphocytes, eosinophils, neutrophils and basophils and combinations thereof.

81. The method of claim 80, wherein the neutrophils are polymorphonuclear leukocytes.

82. The method of claim 73, wherein the transporter is MRP-2.

83. The method of claim 82, wherein the inhibitor is ethacrynic acid.

84. The method of claim 84, wherein the inhibitor is probenecid.

85. A method of treating inflammation in a subject in need thereof comprising administering to the subject a pharmaceutical composition comprising an inhibitor of hepoxilin A3 secretion in an amount sufficient to reduce inflammatory cell accumulation.

86. The method of claim 85, wherein the inhibitor reduces the activity of a hepoxilin A3 transporter.

87. The method of claim 86, wherein the transporter is a MDR protein pump.

88. The method of claim 87, wherein the inhibitor is cyclosporine A.

89. The method of claim 87, wherein the inhibitor is verapamil.

90. The method of claim 87, wherein the inhibitor is ethacrynic acid.

91. The method of claim 87, wherein the inhibitor is probenecid.

92. The method of claim 85 wherein the subject is a human.

93. The method of claim 85, wherein the inflammatory cells are selected from the group consisting of monocytes, lymphocytes, eosinophils, neutrophils and basophils and combinations thereof.

94. The method of claim 93, wherein the neutrophils are polymorphonuclear leukocytes.

95. The method of claim 86, wherein the transporter is MRP-2.

96. The method of claim 95, wherein the inhibitor is ethacrynic acid.

97. The method of claim 95, wherein the inhibitor is probenecid.
